(19) United States
(12) Patent Application Publication
(10) Pub. No.: US 2025/0195372 A1
(43) Pub. Date: Jun. 19, 2025

(54) EMULSION COMPOSITIONS CONTAINING BIS-ETHYLHEXYLOXYPHENOL METHOXYPHENYL TRIAZINE AND SEMI-CRYSTALLINE POLYMER

(71) Applicant: L'OREAL, Paris (FR)

(72) Inventor: Sang Lee Bang, Jersey City, NJ (US)

(21) Appl. No.: 18/543,702

(22) Filed: Dec. 18, 2023

(51) Int. Cl. A61K 8/49; A61K 8/06; A61K 8/81; A61Q 17/04

(52) U.S. Cl. CPC A61K 8/4966; A61K 8/062; A61K 8/8152; A61Q 17/04; A61K 2800/30; A61K 2800/51

(57) ABSTRACT

The present disclosure relates to compositions including 2,4-bis-{[4-(2-ethyl hexyloxy)-2-hydroxy]-phenyl}-6-(4-methoxyphenyl)-1,3,5-triazine and at least one semi-crystalline polymer, as well as to methods of making and using such compositions.

### FIELD OF THE INVENTION

The present disclosure relates to compositions comprising 2,4-bis-{[4-(2-ethyl hexyloxy)-2-hydroxy]-phenyl}-6-(4-methoxyphenyl)-1,3,5-triazine (INCI name: bis-ethylhexyloxyphenol methoxyphenyl triazine) and at least one semi-crystalline polymer, as well as to methods of making and using such compositions.

### DISCUSSION OF BACKGROUND

Radiation of wavelengths between 290 nm and 400 nm allows tanning of the human epidermis, while radiation of wavelengths between 290 and 320 nm, called UVB rays, hinders the development of a natural tan. Exposure is also likely to lead to a detrimental change in the biomechanical properties of the epidermis, resulting in the appearance of wrinkles leading to premature aging of the skin (i.e. photoaging).

UVA rays with wavelengths between 320 and 400 nm penetrate deeper into the skin than UVB rays. UVA rays cause immediate and persistent browning of the skin. Daily exposure to UVA rays, even for a short time, under normal conditions can damage collagen fibers and elastin, resulting in a change in the microrelief of the skin, the appearance of wrinkles and uneven pigmentation (spots, lack of uniformity of complexion).

Many studies show the need for effective protection against UVA and UVB to prevent sunburn, photoaging, and the like.

In order to obtain a high protection product, it is generally necessary to combine a large number of sunscreens and/or a high amount of UV filters to achieve high levels of filtering efficiency.

However, high levels of UV filters do not lend themselves to an easy elaboration of compositions, particularly emulsions, with a stabilized and pleasant texture. However, high levels of UV filters do not lend themselves to an easy elaboration of compositions with a stabilized and pleasant texture.

There remains a need in the art for improved consumer care compositions which possess organic UV filters and have pleasant application properties (e.g., non-greasy feeling) and UV protection properties.

Accordingly, one aspect of the present disclosure is a composition which possesses organic UV filters and has pleasant application properties (e.g., non-greasy feeling) and UV protection properties.

### SUMMARY OF THE INVENTION

The present disclosure relates to emulsion compositions (emulsions) comprising bis-ethylhexyloxyphenol methoxyphenyl triazine (BEMT or bemotrizinol) and at least one semi-crystalline polymer. Preferably the emulsions are in the form of oil-in-water emulsion. Preferably, the compositions further comprise at least one surfactant selected from the group consisting of alkylpolyglucosides, polyol fatty acid esters, anionic carboxylic surfactants or carboxylates, and mixtures thereof. Preferably, the compositions further comprise at least one salt chelating agent.

The present disclosure also relates to methods of treating, caring for, protecting, enhancing the appearance of, and/or making up a keratinous material comprising applying compositions of the present disclosure to a keratinous material in an amount sufficient to treat, care for, enhance the appearance of, and/or make up the keratinous material.

The present disclosure also relates to methods of making emulsion compositions comprising combining bis-ethylhexyloxyphenol methoxyphenyl triazine and at least one semi-crystalline polymer during formation of the compositions. Preferably, the compositions are produced in the form of an oil-in-water emulsion. Preferably, the methods further comprise adding at least one surfactant selected from the group consisting of alkylpolyglucosides, polyol fatty acid esters, anionic carboxylic surfactants or carboxylates, and mixtures thereof, during formation of the emulsion compositions. Preferably, the compositions further comprise adding at least one salt chelating agent during formation of the emulsion compositions.

It is to be understood that both the foregoing general description and the following detailed description are exemplary and explanatory only, and are not restrictive of the disclosure.

### DETAILED DESCRIPTION OF THE INVENTION

In the following description and the claims appended hereto, it is to be understood that the terms used have their ordinary and accustomed meanings in the art, unless otherwise specified.

“About” as used herein means within 10% of the indicated number (e.g., “about 10%” means 9%-11% and “about 2%” means 1.8%-2.2%).

“A” or “an” as used herein means “at least one.”

“At least one” means one or more and thus includes individual components as well as mixtures/combinations.

As used herein, all ranges provided are meant to include every specific point and range within, and combination of subranges between, the given ranges. Thus, a range from 1-5 includes specifically the integers within the range 1, 2, 3, 4 and 5, as well as subranges such as and 2-5, 3-5, 2-3, 2-4, 1-4, etc., as well as all fractional numbers within the range such as 1.2, 2.3, 3.4, etc., and subranges including such fractional numbers such as 1.5-3.8, 2-4.3, 4.2-4.9, etc.

“Film former”, “film-forming polymer” or “film-forming agent” as used herein means a polymer or resin which is capable of leaving a film on the substrate to which it is applied, for example, after a solvent accompanying the film former has evaporated, absorbed into and/or dissipated on the substrate.

“Substituted” as used herein, means comprising at least one substituent. Non-limiting examples of substituents include atoms, such as hydrogen atoms or chlorine atoms, as well as functional groups, such as hydroxyl groups, ether groups, alkoxy groups, acyloxyalky groups, oxyalkylene groups, polyoxyalkylene groups, carboxylic acid groups, amine groups, acylamino groups, amide groups, halogen containing groups, ester groups, thiol groups, sulphonate groups, thiosulphate groups, siloxane groups, and polysiloxane groups. The substituent(s) may be further substituted.

“Volatile”, as used herein, means having a flash point of less than about 115° C.

“Non-volatile”, as used herein, means having a flash point of greater than about 115° C.

“Polymer” as used herein means a compound which is made up of at least two monomers.

“Free” or “substantially free” or “devoid of” as it is used herein means that while it is preferred that no amount of the specific component be present in the composition, it is possible to have very small amounts of it in the compositions of the disclosure provided that these amounts do not materially affect at least one, preferably most, of the advantageous properties of the compositions of the disclosure. Thus, for example, “free of colorant” means that an effective amount (that is, more than trace amounts) of colorant(s) is/are omitted from the composition (that is, about 0% by weight), “substantially free of colorants” means that colorant(s) is/are present in amounts not greater than 0.1% by weight, and “devoid of colorant” means that colorant(s) is/are present in amounts not greater than 0.25% by weight, based on the total weight of the composition. The same nomenclature applies for all other ingredients identified throughout the application and in this paragraph such as, for example, specific UV filters and/or surfactants (compositions of the disclosure which are “free of oxybenzone and/or octinoxate,” “substantially free of oxybenzone and/or octinoxate,” and “devoid of oxybenzone and/or octinoxate,” as well as “free of surfactants,” “substantially free of surfactants,” and “devoid of surfactants,” have meanings consistent with the discussion within this paragraph), even if not specifically discussed for each identified ingredient in the application. Discussed examples of the use of such language such as those in this paragraph are intended to be exemplary, not limiting.

“UV filters” as it is used herein means sunscreen active agents approved by a governmental regulatory agency such as the Food and Drug Administration (FDA) in the U.S. or the EU Commission in Europe and includes organic UV filters such as avobenzone, octocrylene, benzophenones, benzotriazoles and merocyanines, as well as mineral UV filters such as zinc oxide (ZnO) and titanium dioxide (TiO₂).

“Keratinous materials” means nails (finger and/or toe nails), skin such as body, face, and eye area, scalp, keratin fibers such as eyelashes, eyebrows, and hair, and mucous membranes such as lips.

“Physiologically acceptable” means compatible with keratinous materials and having a pleasant color, odor and feel, and which does not cause any unacceptable discomfort (stinging or tautness) liable to discourage a consumer from using the composition. Preferably, the pH of the composition is 5 or higher, preferably 6 or higher, preferably 6.5 or higher, and preferably 7 or higher.

“UV protection efficiency” or “filtering efficiency” in the context of the present disclosure, is evaluated from one or more of SPF, UVAPF, Critical Wavelength, and UVA-I/UV ratio.

“SPF” (Sun Protection Factor) measures the level of protection against erythema provided by a composition. The SPF value corresponds to the ratio between the minimum erythemal dosage (MED) measured wearing the composition and the MED measured with bare skin. “SPF” is a known term in the sunscreen art and is defined, for example, in A new substrate to measure sunscreen protection factors throughout the ultraviolet spectrum, J. Soc. Cosmet. Chem., 40, 127-133 (May/June 1989).

The evaluation of the SPF (Sun Protection Factor) can be performed, for example, in vitro with spectrophotometer by Labsphere (North Sutton, NH, USA). In such an evaluation, the plate is the material on which the tested composition is applied. For such an evaluation, polymethylmethacrylate (PMMA) plates can be used. An example of an acceptable protocol is in the process of ISO accreditation under the name ISO Committee Draft 23675.

The evaluation of the Sun Protection Factor (SPF) can also be performed in-vivo according to the ISO 24444:2019 protocol “Cosmetics-Sun protection test methods-In-vivo determination of the sun protection factor (SPF).” Also, it can be determined according to FDA protocols, as described in the document “Labeling and Effectiveness Testing; Sunscreen Drug Products for Over-the-Counter Human Use” published in the US Federal Register on Jul. 5, 2011 (https://www.federalregister.gov/d/2011-14766); 21 C.F.R. Part 352 Subpart D § 352.72, updated and revised by the 2011 publication in the Federal Register.

“UVAPF” (UVA protection factor) relates to an index characterizing the protection against UVA provided by a composition. For example, the UVAPF index can be measured in vivo according to the “PPD” (Persistent Pigment Darkening) method in the ISO-24442:2022 protocol, measuring observed skin color 2 to 4 hours after UVA exposure. Also, it can be determined according to FDA protocols, again as described in 21 C.F.R. Part 352 Subpart D § 352.72 as discussed above in connection with SPF.

The evaluation of UVA protection can also be measured in vitro with the Labsphere® spectrophotometer under conditions such as those discussed above in connection with SPF. ISO 24443:2021 protocol describes such an in vitro method.

FDA broad spectrum testing procedures, in particular “critical wavelength” testing procedures, can also be found at 21 C.F.R. Part 352 Subpart D § 352.72. Also, broad spectrum testing procedures include determining the UVA1/UV ratio as described in “Sunscreen Drug Products for Over-the-Counter Human Use” published in the Federal Register https://www.federalregister.gov/documents/2019/02/26/2019-03019/sunscreen-drug-products-for-over-the-counter-human-use.” In the assay outlined in the monograph, known as the Boots adaptation of the Diffey/Robson test method, a ratio is generated of the protection afforded by the sunscreen product from UVA1 (340-400 nm) compared to the protection from total UV radiation (UVB and UVA at 290-400 nm) calculated from the absorbance curve. This ratio UVA1/UV would represent the score for the product in the in vitro test.

According to the present disclosure, compositions of the present disclosure preferably have one or more of the following properties:
Compositions have a critical wavelength as determined by FDA's critical wavelength procedures of at least 370 nm;
Compositions have an SPF value of at least 15, preferably at least 30, preferably at least 50 and preferably at least 70;
Compositions have a UVAPF/SPF ratio of at least 1/3, and preferably at least 2/5; and/or
Compositions have a UVA1/UV ratio of 0.7 or higher, preferably 0.75 or higher, and preferably 0.8 or higher.

“Makeup Result” as used herein relates to the visual impact of a composition when applied to a keratinous material such as the skin. The makeup result may relate to the apparent color of the product after application on a keratinous material or an optical effect such as shine, blurring of fine lines and imperfections, or hiding effect on pores or blemishes after the product is applied to the keratinous material.

“Long wear” as used herein refers to compositions where color or other apparent properties remains the same or substantially the same as at the time of application, as viewed by the naked eye, after an extended period of time or after a particular stress event such as water immersion or rubbing. “Long wear” may be evaluated by evaluating long wear properties by any method known in the art for evaluating such properties. For example, long wear may be evaluated by a test involving the application of a composition to keratinous material such as skin and evaluating the color of the composition after an extended period of time. For example, the color of a composition may be evaluated immediately following application to keratinous material such as skin and these characteristics may then be re-evaluated and compared after a certain amount of time. Further, these characteristics may be evaluated with respect to other compositions, such as commercially available compositions. “Long wear” may also be evaluated using vitro methods on non-keratinous substrates such as polymethylmethacrylate (PMMA), wherein properties such as color, transparency, or vitro SPF may be evaluated before and after a period of time or stressor such as water immersion, incubation at elevated temperatures, or an abrasive technique is applied.

“Natural” as in the phrase “natural compound” refers to any compound derived directly from a natural substance such as a plant without having undergone any chemical modification.

“Compound of natural origin” refers to any compound derived from a natural compound which has undergone one or more chemical modifications, for example by organic synthesis reaction, without the properties of the natural compound having been modified.

“Synthetic compound” refers to any compound which is not a natural compound or a compound of natural origin.

“Room temperature” means about 20-25° C.

“Atmospheric pressure” means about 760 mmHg, i.e. about 105 pascals.

“UV filter” and “sunscreen agent” are used interchangeably in this application.

“UV efficacy”, “UV protection”, and “UV efficiency” are used interchangeably in this application.

The compositions and methods of the present disclosure can comprise, consist of, or consist essentially of the essential elements and limitations of the disclosure described herein, as well as any additional or optional ingredients, components, or limitations described herein or otherwise useful. For example, the UV (ultraviolet) absorbing system of the compositions of the disclosure can “consist essentially of” bis-ethylhexyloxyphenol methoxyphenyl triazine.

For purposes of the present disclosure, the “basic and novel property” associated with compositions, components and methods which “consist essentially of” identified ingredients or actions is “UVA protection indicated by UVAPF/SPF and UVA1/UV ratios.”

Compositions of the present disclosure may be in any form suitable for use as a personal care composition, such as that of a stick, a paste, a cream, or a spray. These compositions can be used for any personal care purpose in cosmetic and/or dermatological products such as, for example, a sunscreen, a foundation, lip balms, lipsticks, concealers, mascaras, leave-in hair products, etc.

Referred to herein are trade names for materials including, but not limited to, materials such as polymers and optional components. Materials are not intended to be limited by materials described and referenced by a certain trade name herein. Equivalent materials (e.g., those obtained from a different source under a different name or catalog (reference) number) to those referenced by trade name may be substituted and utilized in the methods described and claimed herein.

All percentages and ratios are calculated by weight unless otherwise indicated. All percentages are calculated based on the total weight of a composition unless otherwise indicated. All component or composition levels are in reference to the active level of that component or composition, and are exclusive of impurities, for example, residual solvents or by-products, which may be present in commercially available sources.

All U.S. patents or patent applications disclosed herein are expressly incorporated by reference in their entirety.

### Composition

### Bis-Ethylhexyloxyphenol Methoxyphenyl Triazine

According to the present disclosure, compositions comprising a UV (ultraviolet) absorbing system comprising 4-bis-{[4-(2-ethyl hexyloxy)-2-hydroxy]-phenyl]-6-(4-methoxyphenyl)-1,3,5-triazine (INCI name: Bis-Ethylhexyloxyphenol Methoxyphenyl Triazine) are provided.

According to preferred embodiments, the UV absorbing system comprises at least 5% by weight of the bis-ethylhexyloxyphenol methoxyphenyl triazine, preferably at least 15% by weight of the bis-ethylhexyloxyphenol methoxyphenyl triazine, preferably at least 20% by weight of the bis-ethylhexyloxyphenol methoxyphenyl triazine, preferably at least 30% by weight of the bis-ethylhexyloxyphenol methoxyphenyl triazine, preferably at least 40% by weight of the bis-ethylhexyloxyphenol methoxyphenyl triazine, preferably at least 50% by weight of the bis-ethylhexyloxyphenol methoxyphenyl triazine, preferably at least 70% by weight of bis-ethylhexyloxyphenol methoxyphenyl triazine, preferably at least 80% by weight of the bis-ethylhexyloxyphenol methoxyphenyl triazine, and preferably at least 90% by weight of the bis-ethylhexyloxyphenol methoxyphenyl triazine, with all weights being based on the total weight of the UV absorbing system. “UV absorbing system” contains all organic UV filters and/or mineral UV filters present in the composition.

According to preferred embodiments, bis-ethylhexyloxyphenol methoxyphenyl triazine is present in compositions of the present disclosure in a UV absorbing effective amount such as, for example, from about 0.1% to about 10% by weight with respect to the total weight of the composition, from about 1% to about 10% by weight, from about 2.5% to about 8% by weight, and from about 4% to about 6% by weight, including all ranges and subranges therebetween such as, for example, from about 4.5% to about 10% by weight, and from about 1% to about 8% by weight, from about 1% to about 6% by weight, from about 5.5% to about 8% by weight, from about 5.7% to about 9% by weight, etc., with all weights being based on the total weight of the composition.

### Additional Organic UV Filter(s)

According to preferred embodiments of the present disclosure, compositions comprising a UV absorbing system optionally further comprising at least one additional organic UV filter (in addition to bis-ethylhexyloxyphenol methoxyphenyl triazine) are provided. Such compositions may also optionally further comprise mineral UV filter(s) such as oxides of zinc, oxides of titanium, oxides of cerium, and mixtures thereof, if desired, although the compositions may also be “free of,” “substantially free of,” or “devoid of” mineral UV filter(s).

Additional organic UV filter(s) can be hydrophilic or lipophilic. “Hydrophilic organic UV filter” means a water-soluble organic UV filter or a water-dispersible (in colloidal form) organic UV filter. “Lipophilic organic UV filter” means a UV filter which is dissolved or dispersed in colloidal form in a liquid fatty phase.

Suitable organic UV filters can be selected from the following non-exhaustive list of compounds: cinnamic compounds; anthranilate compounds; Para-aminobenzoic acid compounds; salicylic compounds; dibenzoylmethane compounds; camphor compounds; benzophenone compounds; β,β-diphenylacrylate compounds; triazine compounds different from Bis-Ethylhexyloxyphenol Methoxyphenyl Triazine; benzotriazole compounds; benzalmalonate compounds including those mentioned in U.S. Pat. No. 5,624,663; benzimidazole derivatives; imidazoline compounds; bis-benzoazolyl compounds as described in patents EP669323 and U.S. Pat. No. 2,463,264; methylene bis-(hydroxyphenyl benzotriazole) compounds as described in applications U.S. Pat. Nos. 5,237,071, 5,166,355, GB2303549, DE 197 26 184 and EP893119; benzoxazole compounds as described in patent applications EP0832642, EP1027883, EP1300137 and DE10162844; polymer filters and silicone filters such as those described in particular in application WO-93/04665; dimers derived from -alkylstyrene such as those described in patent application DE19855649; 4,4-diarylbutadienes compounds as described in applications EP0967200, DE19746654, DE19755649, EP-A-1008586, EP1133980 and EP133981 and mixtures thereof. Preferably, the lipophilic organic UV filters are selected from salicylic compounds, dibenzoylmethane compounds, benzylidene camphor compounds; benzophenone compounds; triazine compounds different from Bis-Ethylhexyloxyphenol Methoxyphenyl Triazine; benzotriazole compounds; as well as other categories of compounds identified herein; and mixtures thereof. Specific reference can be made to suitable salicylic compounds including Homosalate (homomentyl salicylate), for example marketed under the trademark “Eusolex HMS” by Rona/EM Industries; and ethylhexyl salicylate, for example marketed under the trademark “Neo Heliopan OS” by Symrise; and glycol salicylate. Other examples of salicylate compounds include phenyl salicylate; dipropyleneglycol salicylate, for example marketed under the trademark “Dipsal” by Scher; and TEA salicylate, for example marketed under the trademark “Neo Heliopan TS” by Symrise.

Examples of suitable β,β-Diphenylacrylate compounds include Octocrylene, for example marketed under the trademark “Uvinul N539” by BASF; and Etocrylene, for example marketed under the trademark “Uvinul N35” by BASF.

Suitable anthranilic compounds can include menthyl anthranilates, for example marketed under the trademark “Neo Heliopan MA” by Symrise.

Examples of dibenzoylmethane compounds include Butyl methoxydibenzoylmethane, for example marketed under the trademark “Parsol 1789” by DSM; and isopropyl dibenzoylmethane.

Suitable cinnamic compounds include Ethylhexyl methoxycinnamate, for example marketed under the trademark “Parsol MCX” by DSM; isopropyl methoxycinnamate; isopropoxy methoxycinnamate; isoamyl methoxycinnamate, for example marketed under the trademark “Neo Heliopan E 1000” by Symrise; cinoxate (2-ethoxyethyl-4-methoxy cinnamate); DEA methoxycinnamate; diisopropyl methylcinnamate; and glyceryl ethylhexanoate dimethoxycinnamate.

Examples of camphor compounds include benzylidenecamphor derivatives: 3-benzylidene camphor, for example marketed under the trademark “Mexoryl SD” by Chimex; 4-methylbenzylidene camphor, for example marketed under the trademark “Eusolex 6300” by Merck; benzylidene camphor sulfonic acid, for example marketed under the trademark “Mexoryl SL” by Noveal; camphor benzalkonium methosulfate, for example marketed under the trademark “Mexoryl SO” by Noveal; terephthalylidene dicamphor sulfonic acid, for example marketed under the trademark “Mexoryl SX” by Noveal; and polyacrylamidomethyl benzylidene camphor, for example marketed under the trademark “Mexoryl SW” by Noveal.

Suitable benzophenone compounds include benzophenone-1 (2,4-dihydroxybenzophenone), such as that marketed under the trademark “Uvinul 400” by BASF; benzophenone-2 (Tetrahydroxybenzophenone), such as that marketed under the trademark “Uvinul D50” by BASF; Benzophenone-3 (2-hydroxy-4-methoxybenzophenone) or oxybenzone, such as that marketed under the trademark “Uvinul M40” by BASF; benzophenone-4 (hydroxymethoxy benzophonene sulfonic acid), such as that marketed under the trademark “Uvinul MS40” by BASF; benzophenone-5 (Sodium hydroxymethoxy benzophenone Sulfonate); benzophenone-6 (dihydroxy dimethoxy benzophenone); such as that marketed under the trademark “Helisorb 11” by Norquay; benzophenone-8, such as that marketed under the trademark “Spectra-Sorb UV-24” by American Cyanamid; benzophenone-9 (Disodium dihydroxy dimethoxy benzophenonedisulfonate), such as that marketed under the trademark “Uvinul DS-49” by BASF; and benzophenone-12, and n-hexyl 2-(4-diethylamino-2-hydroxybenzoyl)benzoate (such as that marketed under the tradename UVINUL A+ by BASF).

Examples of triazine compounds include Diethylhexyl butamido triazone, such as that marketed under the trademark “Uvasorb HEB” by Sigma 3V; 2,4,6-tris(dineopentyl 4′-aminobenzalmalonate)-s-triazine, bis-ethylhexyloxyphenol methoxyphenyl triazine, such as that marketed under the trademark “TINOSORB S” by BASF, and ethylhexyl triazone, such as that marketed under the trademark “UVTNUL T150” by BASF.

Suitable benzotriazole compounds include phenylbenzotriazole derivatives: 2-(2H-benzotriazole-2-yl)-6-dodecyl-4-methylpheno, branched and linear; and those described in U.S. Pat. No. 5,240,975.

Suitable benzalmalonate compounds include Dineopentyl 4-methoxybenzalmalonate, and polyorganosiloxane comprising benzalmalonate functional groups, such as polysilicone-15, such as that marketed under the trademark “Parsol SLX” by Hoffmann-LaRoche.

Examples of benzimidazole compounds include, in particular, phenylbenzimidazole derivatives such as phenylbenzimidazole sulfonic acid, such as that marketed in particular under the trademark “Eusolex 232” by Merck, and disodium phenyl dibenzimidazole tetrasulfonate, such as that marketed under the trademark “Neo Heliopan AP” by Symrise.

Suitable imidazoline compounds include Ethylhexyl dimethoxybenzylidene dioxoimidazoline propionate.

Examples of bis-benzoazolyl compounds include the compounds described in EP-669,323 and U.S. Pat. No. 2,463,264.

Suitable para-aminobenzoic acid compounds include PABA (p-aminobenzoic acid), ethyl PABA, Ethyl dihydroxypropyl PABA, pentyl dimethyl PABA, ethylhexyl dimethyl PABA, such as that marketed under the trademark “Escalol 507” by ISP, glyceryl PABA, and PEG-25 PABA, such as that marketed under the trademark “Uvinul P25” by BASF.

Suitable methylene bis-(hydroxyphenylbenzotriazol) compounds include 2,2′-methylenebis[6-(2H-benzotriazol-2-yl)-4-methyl-phenol], such as that marketed under the trademark “Mixxim BB/200” by Fairmount Chemical, 2,2′ methylenebis[6-(2H-benzotriazol-2-yl)-4-(1,1,3,3-tetramethylbutyl)phenol], such as that marketed in the micronized form in aqueous dispersion under the trademark “Tinosorb M” by BASF, or under the trademark “Mixxim BB/100” by Fairmount Chemical, and the derivatives as described in U.S. Pat. Nos. 5,237,071 and 5,166,355, GB-2,303,549, DE-197,26,184, and EP-893,119, and Drometrizole trisiloxane, such as that marketed under the trademark “Silatrizole” by Rhodia Chimie or—“Mexoryl XL” by L'Oréal.

Examples of benzoxazole compounds include 2,4-bis[5-I(dimethylpropyl)benzoxazol-2-yl-(4-phenyl)imino]-6-(2-ethylhexyl)imino-1,3,5-triazine, such as that marketed under the trademark of Uvasorb K2A by Sigma 3V.

Suitable examples of screening polymers and screening silicones include the silicones described in WO 93/04665.

Suitable dimers derived from a-alkylstyrene include the dimers described in DE-19855649.

Examples of 4,4-Diarylbutadiene compounds include 1,1-dicarboxy(2,2′-dimethylpropyl)-4,4-diphenylbutadiene.

According to preferred embodiments, compositions of the present disclosure further comprise at least one additional organic UV filter selected from the group consisting of Avobenzone (butyl methoxydibenzoylmethane), Octisalate (ethylhexyl salicylate), Ensulizole (Phenylbenzimidazole sulfonic acid), Homosalate, Octocrylene, and mixtures thereof. In such embodiments, the UV absorbing system can “consist of” or “consist essentially of” (1) bis-ethylhexyloxyphenol methoxyphenyl triazine and (2) at least one organic UV filter selected from the group consisting of Avobenzone (butyl methoxydibenzoylmethane), Octisalate (ethylhexyl salicylate), Ensulizole (Phenylbenzimidazole sulfonic acid), Homosalate, Octocrylene, and mixtures thereof.

According to other preferred embodiments, however, compositions of the present disclosure are “free of,” “substantially free of,” or “devoid of” as defined above of one or more of additional organic UV filters selected from the group consisting of Avobenzone (butyl methoxydibenzoylmethane), Octisalate (ethylhexyl salicylate), Ensulizole (Phenylbenzimidazole sulfonic acid), Homosalate, and Octocrylene, preferably two or more, preferably three or more, preferably four or more, or preferably all five of these sunscreen agents.

According to preferred embodiments, compositions of the present disclosure are “free of,” “substantially free of,” or “devoid of” as defined above of one or more of additional organic UV filters selected from the group consisting of OXYBENZONE (benzophenone-3), OCTINOXATE (Ethylhexyl methoxycinnamate), ETHYLHEXYL TRIAZONE, DROMETRIZOLE TRISILOXANE, METHYLENE BIS-BENZOTRIAZOLYL TETRAMETHYLBUTYL PHENOL, DIETHYLAMINO HYDROXY BENZOYL HEXYL BENZOATE, DIETHYLHEXYL BUTAMIDO TRIAZONE, ISOAMYL P-METHOXYCINNAMATE, POLYSILICONE-15, 4-METHYLBENZYLIDENE CAMPHOR, DISODIUM PHENYL DIBENZIMIDAZOLE TETRASULFONATE, METHOXYPROPYLAMINO CYCLOHEXENYLIDENE ETHOXYETHYLCYANOACET ATE, preferably two or more, preferably three or more, preferably four or more, etc., and preferably “free of,” “substantially free of,” or “devoid of” all of these sunscreen agents.

According to preferred embodiments, compositions of the present disclosure are “free of,” “substantially free of,” or “devoid of” as defined above of OXYBENZONE (benzophenone-3) and/or OCTINOXATE (Ethylhexyl methoxycinnamate).

Preferably, if present, the at least one additional organic UV filter is preferably present in compositions of the present disclosure in an amount of at least about 1% by weight, preferably at least about 5% by weight, preferably at least about 10% by weight, preferably at least about 12.5% by weight, and preferably at least about 15% by weight, with the upper end of the range of additional UV filter present preferably being about 40% by weight (e.g., about 1-40%, about 10-40%, about 12.5-40%, etc.), preferably about 30% by weight (e.g., about 5-30%, about 10-30%, about 15-30%, etc.), preferably about 25% by weight (e.g., about 5-25%, about 10-25%, about 15-25%, etc.), and preferably about 20% by weight (e.g., about 1-20%, about 5-20%, about 10-20%, etc.), with all weights being based on the total weight of the composition.

According to preferred embodiments, bis-ethylhexyloxyphenol methoxyphenyl triazine and the at least one additional organic UV filter, if present, are present in compositions of the present disclosure in a weight % ratio of preferably about 10:1 to about 1:30, preferably about 5:1 to about 1:25, preferably about 3:1 to about 1:20, and preferably about 2:1 to about 1:15, including all ranges and subranges therebetween, such as for example 1:30 to 1:10, 1:20 to 1:5, 1:20 to 1:10, etc, with all weights being based on the weight % of bis-ethylhexyloxyphenol methoxyphenyl triazine and weight % of additional organic UV filter present in the composition. Preferably, a higher weight % of additional organic UV filter is present in the compositions than of bis-ethylhexyloxyphenol methoxyphenyl triazine.

According to preferred embodiments, compositions of the present disclosure comprise 10% or less by weight relative to the total weight of composition of additional UV filters (including mineral UV filters), preferably less than 7.5% by weight relative to the total weight of composition, preferably less than 5% by weight relative to the total weight of composition, preferably less than 3% by weight relative to the total weight of composition, and preferably less than 1% by weight relative to the total weight of composition.

According to preferred embodiments, compositions of the present disclosure comprise 10% or less by weight relative to the total weight of composition of additional mineral UV filters, preferably less than 7.5% by weight relative to the total weight of composition, preferably less than 5% by weight relative to the total weight of composition, preferably less than 3% by weight relative to the total weight of composition, and preferably less than 1% by weight relative to the total weight of composition.

According to preferred embodiments, the UV absorbing system of compositions of the present disclosure can “consist of” or “consist essentially of” (1) bis-ethylhexyloxyphenol methoxyphenyl triazine and (2) additional organic UV filters.

According to preferred embodiments, this disclosure contemplates omitting one or more of any of the specific UV filters discussed above from the UV absorbing system of compositions of the present disclosure. By way of example, octocrylene and/or octinoxate can be omitted from the compositions. Similar omission of one or more of any of the specific UV filters discussed is thus contemplated.

### Semi-Crystalline Polymer

According to the present disclosure, compositions comprising at least one semi-crystalline polymer are provided. “Semi-crystalline polymer” as used herein means polymer(s) comprising a crystallizable part, pendant chain or sequence in the polymer backbone (skeleton), and an amorphous part in the polymer backbone (skeleton), wherein the semi-crystalline polymer has a temperature change of reversible phase of the first order, in particular fusion (solid-liquid transition).

When the crystallizable part is a sequence of the polymeric skeleton, this crystallizable sequence is of a chemical nature different from that of the amorphous sequences; the semi-crystalline polymer is in this case preferably a block polymer, for example of the diblock, triblock or multiblock type, comprising at least one crystallizable sequence and at least one amorphous sequence. By “sequence,” it is meant that at least 5 identical repetition patterns exist. The crystallizable sequence(s) are of a chemical nature different from the amorphous sequence(s).

Preferably, the at least one semi-crystalline polymer has an average molecular mass (Mn) greater than or equal to approximately 2000 g/mol, preferably from about 2,000 to 800,000 g/mol, preferably from about 3,000 to 500,000 g/mol, preferably from about 4,000 to 150,000 g/mol, and preferably about 4,000 to 99,000 g/mol.

Apart from the crystallizable chains or sequences, the at least one semi-crystalline polymer contains chains or sequences that are amorphous. “Crystallizable chain or sequence” as used herein means a chain or sequence which, if it were alone, would pass from the amorphous state to the crystalline state, reversibly, depending on whether it is above or below the melting temperature.

“Chain” as used herein refers to a group of atoms, pendant or lateral to the backbone (skeleton) of the polymer. “Sequence” as used herein refers to a group of atoms belonging to the backbone (skeleton), and a group constituting one of the repeating units of the polymer.

According to preferred embodiments, the at least one semi-crystalline polymer contains a “crystallizable pendant chain” comprising at least 6 carbon atoms, preferably at least 8 carbon atoms, preferably at least 12 carbon atoms, and preferably at least 16 carbon atoms.

Preferably, the polymer skeleton of the at least one semi-crystalline polymer is soluble in the oily phase of the compositions of the present disclosure and introduced into the oily phase during preparation of the compositions of the present disclosure.

Preferably, the at least one semi-crystalline polymer comprises at least one alkyl acrylate chain. Preferably, the crystallizable sequences or chains of such semi-crystalline polymers containing at least one alkyl acrylate chain represent at least about 30% by weight and preferably at least about 40% by weight relative to the total weight of the semi-crystalline polymer.

Preferably, the at least one semi-crystalline polymer with crystallizable sequences are block or multi-block polymers. They can be obtained by polymerization of monomers with reactive (or ethylenic) double bonds or by polycondensation.

Preferably, when the at least one semi-crystalline polymer has crystallizable side chain(s), such polymer(s) are preferably in random or statistical form.

The at least one semi-crystalline polymer can be of synthetic origin. Preferably, the polymer(s) do not contain a polysaccharide skeleton.

The at least one semi-crystalline polymer may or may not be partially crosslinked. If crosslinked, it may involve chemical crosslinking, by reaction with a multifunctional monomer during polymerization. It can also be a physical crosslinking which can then be due either to the establishment of hydrogen or dipolar type bonds between groups carried by the polymer such as the dipolar interactions between carboxylate ionomers, these interactions being in small quantity and carried by the polymer backbone; or due to a phase separation between the crystallizable sequences and the amorphous sequences, carried by the polymer. Preferably, the at least one semi-crystalline polymer is non-crosslinked.

As indicated above, the compositions according to the present disclosure preferably comprise at least one semi-crystalline polymer containing at least one alkyl acrylate chain. Suitable semi-crystalline polymers include, but are not limited to, those resulting from the polymerization of one or more saturated alkyl (meth)acrylates. Preferably, semi-crystalline polymers with a crystallizable side chain are alkyl(meth)acrylate homopolymers, or copolymers of these monomers with a hydrophilic monomer.

According to preferred embodiments, the semi-crystalline polymer containing at least one alkyl acrylate chain comes from a monomer with a crystallizable chain chosen from saturated alkyl (meth)acrylates, preferably having a C16-C-22 chain, such as for example poly(stearyl acrylate) or poly(behenyl acrylate).

According to preferred embodiments, the semi-crystalline polymer containing at least one alkyl acrylate chain is chosen from the copolymers resulting from the polymerization of at least one monomer with a crystallizable chain chosen from the saturated alkyl (meth)acrylates, preferably having a C16-C-22 chain.

According to preferred embodiments, the semi-crystalline polymer containing at least one alkyl acrylate chain is chosen from the homopolymers obtained by polymerization of a monomer chosen from alkyl acrylates and alkyl methacrylates and among the copolymers obtained by copolymerization of a monomer chosen from alkyl acrylates and alkyl methacrylates, with a hydrophilic monomer, preferably N-vinylpyrrolidone or a hydroxyethyl (meth)acrylate, and their mixtures, and preferably a hydroxyethyl (meth)acrylate, and mixtures thereof.

Suitable examples of acceptable semi-crystalline polymers include but are not limited to homopolymers obtained by polymerization of a monomer chosen from alkyl acrylates and alkyl methacrylates, preferably having a C16-C-22 chain such as stearyl acrylates and behenyl acrylates. Specific examples include particular polymers having the name INCI Poly C10-30 Alkyl Acrylate such as the Intelimer®-products from the company EVONIK such as the product Intelimer® IA 13-1 which is a stearyl polyacrylate or the Intelimer® IPA 13-6 product which is a behenyl polymer (behenyl polyacrylate), or the Tego® SP products from the EVONIK Company.

Other suitable examples include, for example, copolymers obtained by copolymerization of a monomer chosen from alkyl acrylates and alkyl methacrylates, with a hydrophilic monomer, preferably a hydroxyethyl (meth)acrylate, such as those described in applications US 2020/0281829 and US 2020/0330334, the entire contents of which are incorporated by reference. Preferably, the copolymer is obtained by copolymerization of at least one monomer chosen from alkyl acrylates, preferably from behenyl acrylates, stearyl acrylates, and mixtures thereof, with a hydrophilic monomer such as hydroxyethyl (meth)acrylate and N-vinylpyrrolidone and preferably hydroxyethyl acrylate.

Preferably, the semi-crystalline polymer containing at least one alkyl acrylate chain is a copolymer of behenyl acrylate/2-hydroxyethyl acrylate, a copolymer of stearyl acrylate/2-hydroxyethyl acrylate and/or one of their mixtures, and in particular the polymer with the INCI name: Poly C12-22 Alkyl Acrylate/Hydroxyethylacrylate Copolymer, in particular marketed under the name Tego SP Senstar the EVONIK Company.

According to preferred embodiments, the semi-crystalline polymer(s) containing at least one alkyl acrylate chain is/are chosen from a stearyl polyacrylate, a behenyl polyacrylate, an acrylate copolymer of behenyl/2-hydroxyethyl acrylate, a copolymer of stearyl acrylate/2-hydroxyethyl acrylate, and mixtures thereof, in particular chosen from the polymer of the name INCI: Poly C10-30 Alkyl Acrylate, the copolymer of the name INCI C12-22 Alkyl Acrylate/Hydroxyethylacrylate Copolymer, and their mixtures, more particularly the polymer with the INCI name: C12-22 Alkyl Acrylate/Hydroxyethylacrylate Copolymer.

According to preferred embodiments, the at least one semi-crystalline polymer contains monomeric units of formulas (A) and (B):

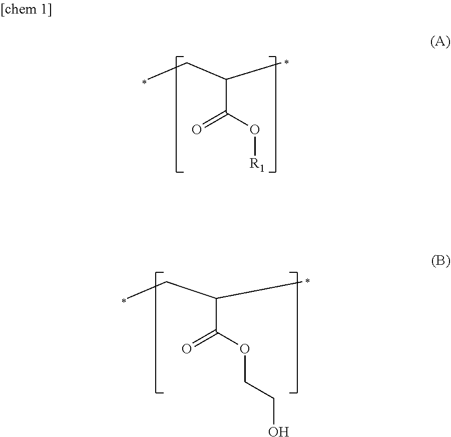

in which:
R1, independently of each other, is chosen from alkyl or alkenyl radicals, and at least 60% by weight of the R1 groups are radicals chosen from stearyl and behenyl radicals, the percentage by weight relating to the sum of all the R1 groups present in the polymer, and the weight ratio of the sum of all the hydroxyethyl acrylate units to the sum of all the acrylate units carrying the R1 group ranges from 1:30 to 1:1, and the sum of the total of units A and B is at least 95% by weight of the total weight of the polymer.

Preferably, R1 consists of alkyl radicals, preferably C16-C22 alkyl radicals, and preferably stearyl radicals (C18) or behenyl radicals (C22).

Preferably, at least 70% by weight of the R1 groups are stearyl or behenyl radicals, preferably at least 80% by weight, and preferably at least 90% by weight.

According to preferred embodiments, all the R1 groups are behenyl radicals.

According to preferred embodiments, all the R1 groups are stearyl radicals.

Preferably, the weight ratio of the sum of all the hydroxyethyl acrylate units to the sum of all the acrylate units carrying the group R1 ranges from 1:15 to 1:1, and preferably ranges from 1:10 to 1:4.

Preferably, the polymeric units present in the polymer consist of the units (A) and (B) described above.

The polymer preferably has a number-average molecular weight Mn ranging from 2000 to 9000 g/mol, preferably from 5000 to 9000 g/mol. The number-average molecular weight may be measured via the gel permeation chromatography method, for example according to the method described below.

The sample is prepared by preparing a solution of the polymer at 10 mg/ml in tetrahydrofuran. The sample is placed in an oven at 54° C. for 10 minutes and then in an oscillating shaker for 60 minutes to aid dissolution. After visual inspection, the sample appeared to be totally dissolved in the solvent.

The sample prepared is analyzed using two polypore 300×7.5 mm columns manufactured by Agilent Technologies, a Waters 2695 chromatographic system, a tetrahydrofuran mobile phase and detection by refractive index. The sample is filtered through a 0.45 μm nylon filter, before being injected into the liquid chromatograph. The standards used for the calibration are the Easi Vial narrow polystyrene (PS) standards from Agilent Technologies. Polystyrene standards ranging from 2 520 000 to 162 daltons can be used for the calibration.

The system is equipped with a PSS SECcurity 1260 RI detector. The polystyrene calibration curve is used to determine the average molecular weight. The recording of the diagrams and the determination of the various molecular weights can be performed by the Win GPC Unichrom 81 program.

Preferably, the polymer has a melting point ranging from 40° C. to 70° C., preferably from 45° C. to 67° C. The melting point is measured by differential scanning calorimetry (DSC), for example according to methods based on standards ASTM E791 and ASTM D 34182 and the DSC calibration is performed according to standard ASTM E 9672.

According to preferred embodiments, when the polymer is such that at least 60% by weight of the R₁ groups are stearyl radicals, then the polymer preferably has a melting point of from 40° C. to 60° C., preferably from 45° C. to 55° C.

According to preferred embodiments, when the polymer is such that at least 60% by weight of the R₁ groups are behenyl radicals, then the polymer has a melting point of from 60° C. to 70° C., preferably from 63° C. to 67° C.

The polymer of the present disclosure can be prepared by polymerization of a monomer having the formula below:

CH₂═CH—COO—R₁,
R₁ having the meaning previously described, and of 2-hydroxyethyl acrylate.

The polymerization may be performed according to known methods, such as solution polymerization or emulsion polymerization.

The polymerization is, for example, described in document US 2007/0264204, the entire contents of which is hereby incorporated by reference.

According to preferred embodiments, the at least one semi-crystalline polymer is present in compositions of the present disclosure in an amount (active material) of from 0.05 to 10% by weight, relative to the total weight of the composition, preferably from 0.1 to 5% by weight, and preferably from 0.2 to 2% by weight, including all ranges and subranges therebetween.

### Galenic Form

Compositions of the present disclosure may be in any form suitable for use as a personal care composition, such as that of a stick, a paste, a cream, an anhydrous composition, an emulsion (oil-in-water, water-in-oil, multiple emulsion such as oil-in-water-in-oil) including in nanoemulsion form, a gel, a liquid, a solid, a mousse, a spray, etc. Preferably, the composition is in the form of an emulsion, preferably an oil-in-water emulsion (O/W). “Emulsion” as used herein means any macroscopically homogeneous kinetically stable composition comprising at least two phases that are immiscible with each other; one being the continuous dispersing phase and the other being dispersed in said continuous phase in the form of droplets. The two phases are generally stabilized kinetically by at least one emulsifying system which may comprise at least one emulsifying surfactant (emulsifier or surfactant). A distinction can be made between emulsions of the oil-in-water type, called “direct” emulsions, containing a continuous aqueous dispersing phase and a discontinuous dispersed oily phase, and emulsions of the water-in-oil (W/O) type called “inverse” emulsions, containing a phase continuous oily dispersant phase and a discontinuous aqueous dispersed phase. There are also multiple emulsions such as water-in-oil-in-water or oil-in-water-in-oil emulsions.

The composition according to the present disclosure may comprise at least one emulsifier chosen from amphoteric, anionic, cationic or non-ionic emulsifiers, used alone or in a mixture. The emulsifiers are chosen appropriately depending on the emulsion to be obtained (W/O or O/W).

Suitable examples of nonionic W/O emulsifying surfactants include but are not limited to alkyl esters or ethers of sorbitan, glycerol, polyol, glycerol or sugars; silicone surfactants such as emulsifying silicone elastomers such as pegylated and/or alkoxylated dimethicone crosspolymers such as dimethicone/peg 10/15 crosspolymer, dimethicone copolyols such as the mixture of cyclomethicone and dimethicone copolyol, sold under the name “DC 5225 C®” by the company Dow Corning, and alkyl-dimethicone copolyols such as Laurylmethicone copolyol sold under the name “Dow Corning 5200 Formulation Aid” by the Dow Corning Company; Cetyl dimethicone copolyol such as the product sold under the name Abil EM 90R® by the company Goldschmidt and the mixture of cetyl dimethicone copolyol, polyglycerol isostearate (4 moles) and hexyl laurate sold under the name ABIL WE O9® by the Goldschmidt company. One or more co-emulsifiers can also be added, which, advantageously, can be chosen from the group comprising alkylated polyol esters. Specific examples include but are not limited to specific polyol alkyl esters such as polyethylene glycol esters such as PEG-30 Dipolyhydroxystearate such as the product marketed under the name Arlacel P135® by the company ICI; specific glycerol and/or sorbitan esters such as polyglycerol isostearate, such as the product marketed under the name Isolan GI 34® by the company Goldschmidt; sorbitan isostearate, such as the product marketed under the name Arlacel 987® by the company ICI; sorbitan isostearate and glycerol, such as the product sold under the name Arlacel 986® by the company ICI; and mixtures thereof.

Suitable examples of nonionic O/W emulsifying surfactants include but are not limited to polyoxyalkylenated fatty acid and glycerol esters (more particularly polyoxyethylenated and/or polyoxypropylenated) such as the ester of polyethylene glycol and stearic acid with INCI name PEG-100 STEARATE marketed under the name Myrj S100-PA-(SG) by the company CRODA; oxyalkylenated fatty acid and sorbitan esters; esters of polyoxyalkylenated fatty acids (in particular polyoxyethylenated and/or polyoxypropylenated) optionally in association with a fatty acid and glycerol ester such as the PEG-100 Stearate/Glyceryl Stearate mixture sold for example by the company ICI under the name Arlacel 165; oxyalkylenated fatty alcohol ethers (oxyethylenated and/or oxypropylenated); sugar esters such as sucrose stearate; fatty alcohol and sugar ethers, in particular alkyl polyglucosides (APG) such as C12-C20 alkyl glucosides such as that which is marketed by the company Seppic under the name Montanov L, decylglucoside and laurylglucoside marketed for example by the company Henkel under the respective names Plantaren 2000® and Plantaren 1200®, the cetostearylglucoside optionally mixed with cetostearyl alcohol, marketed for example under the name Montanov 68® by the company Seppic, under the name Tegocare CG90® by the company Goldschmidt and under the name Emulgade KE3302® by the company Henkel, as well as arachidyl glucoside, for example in the form of the mixture of arachidic and behenic alcohols and arachidylglucoside marketed under the name Montanov 202® by the company Seppic. According to a particular embodiment of the invention, the mixture of the alkylpolyglucoside as defined above with the corresponding fatty alcohol can be in the form of a self-emulsifying composition, as described for example in document WO-A-92/06778.

Suitable examples of anionic O/W emulsifying surfactants include but are not limited to amino acids modified by at least one C8-C30 hydrocarbon chain, preferably C8-C24, and their salts, in particular acyl glutamic acids (INCI name: acyl glutamic acid) or one of their salts (acyl glutamates), such as stearoyl glutamic acid or one of its salts, in particular sodium stearoyl glutamate (INCI name). Such compounds are marketed under the name AMISOFT by the company AJINOMOTO and in particular under the references Amisoft CA, Amisoft LA, Amisoft HS 11 PF, Amisoft MK-11, Amisoft LK-11, Amisoft CK-11, or even under the name EUMULGIN SG by the company COGNIS. Further examples of anionic O/W emulsifying surfactants include but are not limited to hydrophobic modified polysaccharides, in particular inulins modified by hydrophobic chains such as alkylcarbamate groups, in particular C8-C18 alkyl carbamate, and more. particularly laurylcarbamate, with the product sold under the name INUTEC SL by the company CREACHEM being an example of a commercial product thereof.

### Nonionic Alkylpolyglycoside Surfactants

According to preferred embodiments, compositions of the present disclosure comprise one or more nonionic surfactants of the alkylpolyglycoside type. Suitable nonionic surfactants of the alkylpolyglycoside type which can be used according to the present disclosure can be chosen from the compounds of the following general formula:

R1O—(R2O)t-(G)v in which:
R1 represents a linear or branched alkyl or alkenyl radical comprising 6 to 24 carbon atoms, in particular 8 to 18 carbon atoms, or an alkylphenyl radical whose linear or branched alkyl radical comprises 6 to 24 carbon atoms, preferably 8 to 18 carbon atoms;
R2 represents an alkylene radical containing 2 to 4 carbon atoms;
G represents a sugar unit comprising 5 to 6 carbon atoms;
t designates a value ranging from 0 to 10, preferably from 0 to 4;
v designates a value ranging from 1 to 15, preferably from 1 to 4.

Preferably, the nonionic surfactants of the alkyl (poly) glycoside type are compounds of formula described above in which:
R1 designates a saturated or unsaturated, linear or branched alkyl radical containing 8 to 18 carbon atoms
R2 represents an alkylene radical containing 2 to 4 carbon atoms,
t designates a value ranging from 0 to 3, preferably equal to 0,
G designates glucose, fructose or galactose, preferably glucose;
the degree of polymerization, that is to say the value of v, which can range from 1 to 15, preferably from 1 to 4; the average degree of polymerization being more particularly between 1 and 2, and preferably from 1.1 to 1.5 on average.

The glucosidic bonds between the sugar units are generally of type 1-6 or 1-4, preferably of type 1-4.

Preferably, the alkyl(poly)glycoside is an alkyl(poly)glucoside.

Preferably, the nonionic surfactants are chosen from (C6-C24 alkyl) polyglycosides, such as coco glucoside, caprylyl/capryl glucoside, lauryl glucoside, decyl glucoside and cetearyl glucoside, and mixtures thereof.

Preferably, the composition comprises one or more nonionic surfactants chosen from (C6-C24 alkyl)(poly) glycosides, alone or in a mixture, and more particularly (C8-C18 alkyl)(poly) glycosides.

Among suitable commercial products, mention can be made of the products sold by the company COGNIS under the names PLANTAREN® (600 CS/U, 1200 and 2000) or PLANTACARE® (818, 1200 and 2000); the products sold by the company SEPPIC under the names ORAMIX CG 110 and ORAMIX® NS 10; products sold by the company BASF under the name LUTENSOL GD 70, products sold by the company CHEM Y under the name AG10 LK, products sold by the company EVONIK GOLDSCHMIDT under the trade names TEGO CARE CG 90 or TEGO CARE CG 90 MB, the products sold by the company SEPPIC under the trade names Montanov® L, Montanov® 68, Montanov® 68 MB, Montanov® 14 or Montanov® 202, or the products sold by the company BASF under the name Emulgade® PL 68/50.

Preferably, if present, the total content of the nonionic surfactant(s) of the alkylpolyglycoside type is from 0.01 to 15% by weight, preferably from 0.1 to 10% by weight, preferably from 0.2 to 8% by weight, preferably from 0.3 to 6% by weight, and preferably from 0.3 to 3% by weight relative to the total weight of the composition.

### Polyol Fatty Acid Ester and Anionic Carboxylic Surfactant or Carboxylate

According to preferred embodiments, compositions of the present disclosure comprise:
at least one ester of C12-C24 fatty acid and C2-C24 polyol, preferably at least one ester of C16-C18 fatty acid and glycerol; and/or
at least one anionic carboxylic or carboxylate surfactant chosen from amino acids modified by at least one C8-C30 hydrocarbon chain, preferably C8-C24, and their salts.

According to preferred embodiments, the C12-C24 fatty acid for the at least one C12-C24 fatty acid ester of a C2-C24 polyol can be a saturated or unsaturated acid such as, for example, stearic acid, oleic acid, palmitic acid, etc.

For the purposes of the present invention, the term “polyol” means any organic molecule including at least two free hydroxyl groups.

According to preferred embodiments, the polyol for the at least one C12-C24 fatty acid ester of a C2-C24 polyol can be a compound of linear, branched or cyclic structure, saturated or unsaturated alkyl type, bearing on the alkyl at least two OH functions, preferably at least three OH functions. Preferably, the polyol for the at least one C12-C24 fatty acid ester of a C2-C24 polyol preferably has 3 to 16 carbon atoms.

Preferably, the polyol is chosen, for example, from the group consisting of ethylene glycol, pentaerythritol, trimethylolpropane, propylene glycol, 1,3-propanediol, butylene glycol, isoprene glycol, pentylene glycol, hexylene glycol, polyglycerin-3, glycerol, and mixtures thereof. Preferably, the polyol is selected from 1,3-propanediol, polyglycerin-3, glycerol, pentylene glycol, and mixtures thereof. Preferably, the polyol is glycerol.

By way of examples of C12-C24 fatty acid esters of glycerol, mention may be made of glyceryl laurate, glyceryl oleate, glyceryl stearate, glyceryl palmitate, and mixtures thereof. As commercially available products, mention may be made of glyceryl stearate, such as the product sold under the name Tegin MO by Evonik Goldschmidt, and glyceryl laurate, such as the product sold under the name Imwitor 312O by Hiils, and monoglyceride stearates (INCI name: Glyceryl Stearate), for example sold under the name Dimodan HP by Danisco, or those sold under the name Tegin 90 by Evonik Goldschmidt.

Preferably, the C12-C24 fatty acid ester of glycerol is glyceryl stearate.

According to preferred embodiments, the at least one C12-C24 fatty acid ester(s) of a C2-C24 polyol is present in compositions of the present disclosure in an amount (active material) of from 0.1% to 2% by weight, and preferably from 0.2% to 0.8% by weight, with all weights being with respect to the total weight of the composition, including all ranges and subranges therebetween.

According to the present disclosure, carboxylic or carboxylate anionic surfactant can be selected from the group consisting of amino acids modified with at least one C₈-C₃₀ hydrocarbon-based chain, preferably a C₈-C₂₄ hydrocarbon-based chain, and salts thereof, are provided. The term “hydrocarbon-based chain” means a linear or branched, saturated or unsaturated, organic group constituted mainly of hydrogen atoms and carbon atoms, in which one or more carbon atoms can be replaced with an oxygen atom or a nitrogen atom. As examples of C₈-C₃₀ hydrocarbon-based chains, mention may be made of C₈-C₃₀ acyl radicals.

Exemplary amino acids include, but are not limited to, alanine, arginine, aspartic acid, glutamic acid, glycine, isoleucine, leucine, lysine, phenylalanine, serine, tyrosine, valine and sarcosine.

More specifically mention may be made of the following compounds: dipotassium capryloyl glutamate, dipotassium undecylenoyl glutamate, disodium capryloyl glutamate, disodium cocoyl glutamate, disodium lauroyl glutamate, disodium stearoyl glutamate, disodium undecylenoyl glutamate, potassium capryloyl glutamate, potassium cocoyl glutamate, potassium lauroyl glutamate, potassium myristoyl glutamate, potassium stearoyl glutamate, potassium undecylenoyl glutamate, sodium capryloyl glutamate, sodium cocoyl glutamate, sodium lauroyl glutamate, sodium myristoyl glutamate, sodium olivoyl glutamate, sodium palmitoyl glutamate, sodium stearoyl glutamate, sodium undecylenoyl glutamate, cocoyl methyl β-alaninate, lauroyl β-alaninate, lauroyl methyl β-alaninate, myristoyl β-alaninate, potassium lauroyl methyl β-alaninate, sodium cocoyl alaninate, sodium cocoyl methyl β-alaninate and sodium myristoyl methyl β-alaninate palmitoyl glycinate, sodium lauroyl glycinate, sodium cocoyl glycinate, sodium myristoyl glycinate, potassium lauroyl glycinate, potassium cocoyl glycinate, potassium lauroyl sarcosinate, potassium cocoyl sarcosinate, sodium cocoyl sarcosinate, sodium lauroyl sarcosinate, sodium myristoyl sarcosinate, sodium oleoyl sarcosinate, sodium palmitoyl sarcosinate ammonium lauroyl sarcosinate, sodium lauroyl aspartate, sodium myristoyl aspartate, sodium cocoyl aspartate, sodium caproyl aspartate, disodium lauroyl aspartate, disodium myristoyl aspartate, disodium cocoyl aspartate, disodium caproyl aspartate, potassium lauroyl aspartate, potassium myristoyl aspartate, potassium cocoyl aspartate, potassium caproyl aspartate, dipotassium lauroyl aspartate, dipotassium myristoyl aspartate, dipotassium cocoyl aspartate, dipotassium caproyl aspartate, and mixtures thereof.

According to preferred embodiments, the anionic carboxylic or carboxylate surfactant(s) are chosen from the compounds of the following structure:

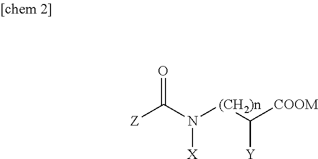

in which:
Z represents a saturated or unsaturated, linear or branched hydrocarbon-based group comprising from 8 to 24 carbon atoms, preferably from 8 to 22 carbon atoms,
X is a hydrogen atom or a methyl group;
n is equal to 0 or to 1;
Y is a group chosen from a hydrogen atom and a —CH3, —CH(CH3)2, —CH2CH(CH3)2, —CH(CH3)CH2CH3, —CH2C6H5, —CH2C2H40H, —CH2OH, —CH(OH)CH3, —(CH2)4NH2, —(CH2)3NHC(NH)NH2, —CH2C(O)O-M+, —(CH2)2C(O)OH and —(CH2)2C(O)O-M+ group; and
M is a hydrogen atom or a cation such as sodium, potassium, ammonium or triethanolamine.

According to preferred embodiments, in formula 2:
Z represents a linear or branched C8-C22 alkyl or alkenyl group;
X is a hydrogen atom or a methyl radical;
n is equal to 0;
Y is a hydrogen atom, a —(CH2)2C(O)OH group or a —(CH2)2C(O)O-M+ group; and
M is a cation such as sodium, potassium, ammonium or triethanolamine.

According to preferred embodiments, the composition according to the present disclosure comprises at least one acyl glutamic acid (INCI name: acyl glutamic acid) or a salt thereof (acyl glutamates). Preferably, the acyl glutamic acid(s) is/are chosen from acyl glutamic acids in which the acyl group comprises from 8 to 30 carbon atoms, preferably from 10 to 24 carbon atoms, and preferably from 12 to 22 carbon atoms, such as, for example, lauroylglutamic acid, myristoylglutamic acid, palmitoylglutamic acid, stearoylglutamic acid, behenoylglutamic acid, olivoylglutamic acid, cocoylglutamic acid, and the salts of these acids, especially the salts of alkali metals such as Na, Li or K, preferably Na or K, the salts of alkaline-earth metals such as Mg, or the ammonium salts of said acids.

Exemplary salts of amino acids modified with at least one C8-C30 hydrocarbon-based chain include, but are not limited to, glutamate salts, and in particular dipotassium capryloyl glutamate, dipotassium undecylenoyl glutamate, disodium capryloyl glutamate, disodium cocoyl glutamate, disodium lauroyl glutamate, disodium stearoyl glutamate, disodium undecylenoyl glutamate, potassium capryloyl glutamate, potassium cocoyl glutamate, potassium lauroyl glutamate, potassium myristoyl glutamate, potassium stearoyl glutamate, potassium undecylenoyl glutamate, sodium capryloyl glutamate, sodium cocoyl glutamate, sodium lauroyl glutamate, sodium myristoyl glutamate, sodium olivoyl glutamate, sodium palmitoyl glutamate, sodium stearoyl glutamate, sodium undecylenoyl glutamate.

Preferably, the acyl glutamic acid(s) or a salt thereof is/are chosen from lauroylglutamic acids, cocoylglutamic acids, sodium stearoyl glutamate, potassium lauroyl glutamate, potassium cocoyl glutamate, sodium olivoyl glutamate and mixtures thereof. Preferably, the acyl glutamic acid or a salt thereof is sodium stearoyl glutamate. Such compounds are sold under the name Amisoft by Ajinomoto and, in particular, under the references Amisoft CA, Amisoft LA, Amisoft HS 11 PF, Amisoft MK-11, Amisoft LK-11 and Amisoft CK-11, or alternatively under the name Eumulgin SG by Cognis.

Mention may also be made of triethanolamine cocoyl glutamate sold under the name Amisoft CT12 by Ajinomoto, and triethanolamine lauroyl glutamate sold under the name Acylglutamate LT-12 by Ajinomoto.

As acyl glutamic acid salts, mention may also be made of sodium hydrogenated tallowoyl glutamate, such as the product sold under the reference Acylglutamate HS 11 by Ajinomoto and disodium hydrogenated tallow glutamate, such as the product sold under the reference Acylglutamate HS-21 by Ajinomoto.

Mention may also be made of commercial mixtures of surfactants comprising at least one glutamic acid derivative or a salt of said derivative, for instance the mixture of acyl glutamate salts such as Amisoft LS-22 sold by Ajinomoto.

According to preferred embodiments of the present disclosure, compositions of the present disclosure comprise the monosodium salt of n-stearoyl-L-glutamic acid (INCI name: sodium stearoyl glutamate), such as the product sold by Ajinomoto under the reference Amisoft HS 11 PF.

According to preferred embodiments, the at least one amino acid modified with at least one C₈-C₃₀ hydrocarbon-based chain, and/or salts thereof, is preferably present in compositions of the present disclosure in an amount of from 0.01% to 5% by weight, preferably from 0.01% to 2% by weight, preferably from 0.05% to 1% by weight, preferably from 0.1% to 0.8% by weight, and preferably from 0.1% to 0.5% by weight, all weights being with respect to the total weight of the composition, including all ranges and subranges therebetween.

### Emulsifying Silicone Surfactants

According to preferred embodiments, compositions of the present disclosure comprise at least one emulsifying silicone elastomer. The term “emulsifying silicone elastomer” means a silicone elastomer comprising at least one hydrophilic chain. The emulsifying silicone elastomer is preferably chosen from polyoxyalkylenated silicone elastomers and polyglycerolated silicone elastomers, and mixtures thereof.

A polyoxyalkylenated silicone elastomer is a crosslinked organopolysiloxane that can be obtained by crosslinking addition reaction of diorganopolysiloxane containing at least one hydrogen bonded to silicon and of a polyoxyalkylene having at least two ethylenically unsaturated groups. Preferably, the polyoxyalkylenated crosslinked organopolysiloxane is obtained by crosslinking addition reaction (A1) of diorganopolysiloxane containing at least two hydrogens each bonded to a silicon, and (B1) of polyoxyalkylene containing at least two ethylenically unsaturated groups, in particular in the presence (C1) of a platinum catalyst, as described, for example, in U.S. Pat. Nos. 5,236,986 and 5,412,004, the entire contents of both of which are hereby incorporated by reference. In particular, the organopolysiloxane can be obtained by reaction of polyoxyalkylene (in particular polyoxyethylene and/or polyoxypropylene) containing dimethylvinylsiloxy end groups and of methylhydrogeno-polysiloxane containing trimethylsiloxy end groups, in the presence of a platinum catalyst.

The organic groups bonded to silicon atoms of the compound (A1) may be alkyl groups containing from 1 to 18 carbon atoms, such as methyl, ethyl, propyl, butyl, octyl, decyl, dodecyl (or lauryl), myristyl, cetyl or stearyl; substituted alkyl groups such as 2-phenyl-ethyl, 2-phenylpropyl or 3,3,3-trifluoropropyl; aryl groups such as phenyl, tolyl or xylyl; substituted aryl groups such as phenylethyl; and substituted monovalent hydrocarbon-based groups such as an epoxy group, a carboxylate ester group or a mercapto group. Compound (A1) may thus be chosen from methylhydrogenopolysiloxanes containing trimethylsiloxy end groups, copolymers of dimethylsiloxane-methylhydrogenosiloxane containing trimethylsiloxy end groups, cyclic dimethylsiloxane-methylhydrogenosiloxane copolymers, and copolymers of dimethylsiloxane-methylhydrogenosiloxane-laurylmethylsiloxane containing trimethylsiloxy end groups.

Compound (C1) is the crosslinking reaction catalyst, and is in particular chloroplatinic acid, chloroplatinic acid-olefin complexes, chloroplatinic acid-alkenylsiloxane complexes, chloroplatinic acid-diketone complexes, platinum black and platinum on a support.

Preferably, polyoxyalkylenated silicone elastomers may be formed from divinyl compounds, in particular polyoxyalkylenes having at least two vinyl groups, reacting with Si—H bonds of a polysiloxane.

Polyoxyalkylenated silicone elastomers can be preferably mixed with at least one hydrocarbon-based oil and/or one silicone oil so as to form a gel. In these gels, the polyoxyalkylenated elastomer can be in the form of non-spherical particles.

Polyoxyalkylenated elastomers are in particular described in U.S. Pat. Nos. 5,236,986, 5,412,004, 5,837,793 and 5,811,487, the entire contents of all of which are hereby incorporated by reference. Polyoxyalkylenated silicone elastomers that may be used include those sold under the names KSG-21, KSG-20, KSG-30, KSG-31, KSG-32, KSG-33, KSG-210, KSG-310, KSG-320, KSG-330, KSG-340 and X-226146 by the company Shin-Etsu, and DC9010 and DC9011 by the company Dow Corning, KSG-210 is particularly notable.

Suitable emulsifying silicone elastomers also include polyglycerolated silicone elastomers. A polyglycerolated silicone elastomer is a crosslinked elastomeric organopolysiloxane that can be obtained by a crosslinking addition reaction of diorganopolysiloxane containing at least one hydrogen bonded to silicon and of polyglycerolated compounds having ethylenically unsaturated groups, in particular in the presence of a platinum catalyst. Preferably, the crosslinked elastomeric organopolysiloxane is obtained by crosslinking addition reaction (A) of diorganopolysiloxane containing at least two hydrogens each bonded to a silicon, and (B) of glycerolated compounds having at least two ethylenically unsaturated groups, in particular in the presence (C) of a platinum catalyst. For example, the organopolysiloxane can be obtained by reaction of a polyglycerolated compound containing dimethylvinylsiloxy end groups and of methylhydrogenopolysiloxane containing trimethylsiloxy end groups, in the presence of a platinum catalyst.

The organic groups bonded to the silicon atoms of compound (A) may be alkyl groups containing from 1 to 18 carbon atoms, such as methyl, ethyl, propyl, butyl, octyl, decyl, dodecyl (or lauryl), myristyl, cetyl or stearyl; substituted alkyl groups such as 2-phenylethyl, 2-phenylpropyl or 3,3,3-trifluoropropyl; aryl groups such as phenyl, tolyl or xylyl; substituted aryl groups such as phenylethyl; and substituted monovalent hydrocarbon-based groups such as an epoxy group, a carboxylate ester group or a mercapto group. Preferably, said organic group is chosen from methyl, phenyl and lauryl groups.

Compound (A) can thus be chosen from methylhydrogenopolysiloxanes containing trimethylsiloxy end groups, copolymers of dimethylsiloxane-methyl-hydrogenosiloxane containing trimethylsiloxy end groups, dimethylsiloxane-methylhydrogenosiloxane cyclic copolymers, and copolymers of dimethylsiloxane-methyl-hydrogenosiloxane-laurylmethylsiloxane containing trimethylsiloxy end groups.

Compound (B) may be a polyglycerolated compound corresponding to formula (B′) below:

CmH2m−1-O-[Gly]n-CmH2m−1
in which m is an integer ranging from 2 to 6; n is an integer ranging from 2 to 200, preferably ranging from 2 to 100, preferably ranging from 2 to 50, preferably ranging from 2 to 20, preferably from 2 to 10, and preferably ranging from 2 to 5, and where Gly denotes:

—CH2-CH(OH)—CH2-O— or —CH2-CH(CH2OH)—O—

Preferably, the sum of the number of ethylenic groups per molecule of compound (B) and of the number of hydrogen atoms bonded to silicon atoms per molecule of compound (A) is at least 4.

Compound (C) is the crosslinking reaction catalyst, and is preferably chosen from catalysts such as chloroplatinic acid, chloroplatinic acid-olefin complexes, chloroplatinic acid-alkenylsiloxane complexes, chloroplatinic acid-diketone complexes, platinum black, or platinum on a support.

Suitable polyglycerolated silicone elastomers can be mixed with at least one hydrocarbon-based oil and/or one silicone oil so as to form a gel. In these gels, the polyglycerolated elastomer is often in the form of non-spherical particles. Such elastomers are in particular described in patent application U.S. Pat. No. 9,090,755, the entire contents of which is hereby incorporated by reference. As polyglycerolated silicone elastomers, mention may be made of those sold under the names KSG-710, KSG-810, KSG-820, KSG-830 and KSG-840 by the company Shin-Etsu.

According to preferred embodiments, the at least one emulsifying silicone elastomer is preferably present in compositions of the present disclosure in an amount of from 0.01% to 5% by weight, preferably from 0.01% to 2% by weight, preferably from 0.05% to 1% by weight, preferably from 0.1% to 0.8% by weight, and preferably from 0.1% to 0.5% by weight, all weights being with respect to the total weight of the composition, including all ranges and subranges therebetween.

### C12-C24 Saturated Fatty Alcohols

According to preferred embodiments, compositions optionally further comprising at least one C12-C24 saturated fatty alcohol are provided. Preferably, the at least one C12-C24 saturated fatty alcohol is linear and/or comprises from 14 to 22 carbon atoms. Suitable examples included, but are not limited to, cetyl alcohol, stearyl alcohol, cetearyl alcohol, myristyl alcohol, lauryl alcohol, tridecyl alcohol, pentadecyl alcohol, hexadecyl alcohol, arachidyl alcohol and/or behenyl alcohol, with cetyl alcohol, stearyl alcohol and/or cetearyl alcohol being most preferred.

Commercially available cetyl alcohol includes, but is not limited to, those sold under the names Ecorol® 16/98 F and Ecorol® 16/98 P by Ecogreen Oleochemicals, Tegoalkanol® 16 by Evonik Goldschmidt, Lanette® 16 by Cognis, Vegarol® 1698 by VVF, Alkonat® 1698 P by Oxiteno, Cetyl Alcohol 98% MIN by Emery Oleochemicals, Ginol® 16 (98%) by Godrej Industries, Nacol® 16-98 by Sasol, Kalcol® 6098 by Kao and Acilol® 16 by Aegis Chemical.

Commercially available stearyl alcohol includes, but is not limited to, those sold under the names Tegoalkanol® 18 by Evonik Goldschmidt, Ecorol® 18/98 F and Ecorol® 18/98 P by Ecogreen Oleochemicals, Lanette® 18 by Cognis, Kalcol® 8098 by Kao, Acilol® 18 by Aegis Chemical, Nacol® 18-98 by Sasol and NAA® 45 by Nihon Yushi.

Commercially available cetearyl alcohol includes, but is not limited to, those sold under the names Ecorol® 68/50 F and Ecorol® 68/50 P by Ecogreen Oleochemicals, Lanette® O OR and Lanette® O OR Flakes by Cognis, Alkonat® 1618 C50 P by Oxiteno, Nafol® 16-18 EN by Sasol, Alcohol Cetoestearilico 50/50 by Industria Quimica Del Centro, Conol® 30 CK by New Japan Chemical, Cetylstearyl Alcohol 50:50 by Evonik Goldschmidt, Kalcol® 6850 by Kao, Vegarol® 1618 (50:50) by VVF and Ginol® 1618 50:50 OR by Godrej Industries.

Commercially available behenyl alcohol includes, but is not limited to, those sold under the names Lanette 22 by BASF.

Preferably, compositions of the present disclosure possess an alpha-gel structure, that is to say that it has a lamella phase, believed to be due to the presence of a surfactant and of a C₁₂-C₂₄ saturated fatty alcohol.

If present, the at least one C₁₂-C₂₄ saturated fatty alcohol is preferably present in compositions of the present disclosure in an amount of from 0.1% to 5% by weight, preferably from 0.2% to 3% by weight, and preferably from 0.5% to 2% by weight, all weights being based on the total weight of the composition, including all ranges and subranges therebetween.

### Waxes

According to preferred embodiments, compositions of the present disclosure optionally further comprise at least one wax. “Wax” means a deformable or undeformable lipophilic compound, which is solid at ambient temperature (25° C.), with a reversible solid/liquid change of state, having a melting point of greater than or equal to 30° C., which may be up to 120° C. In particular, the waxes that are suitable for use in the disclosed compositions preferably have a melting point of greater than or equal to 60° C., preferably greater than or equal to 70° C.

The term “lipophilic compound” refers to a compound having an acid number and a hydroxyl number of less than 150 mg KOH/g.

Melting point corresponds to the temperature of the most endothermic peak observed on thermal analysis (DSC) as described in standard ISO 11357-3; 1999. The melting point of the wax may be measured using a differential scanning calorimeter (DSC), for example the calorimeter sold under the name MDSC 2920 by TA Instruments or the calorimeter sold under the name DSC Q100 by TA Instruments with the TA Universal Analysis software.

A measuring protocol is as follows:

A sample of 5 mg of wax placed in a crucible is subjected to a first temperature ramp passing from −20° C. to 100° C., at a heating rate of 10° C./minute, it is then cooled from 100° C. to −20° C. at a cooling rate of 10° C./minute and is finally subjected to a second temperature ramp passing from −20° C. to 100° C. at a heating rate of 5° C./minute. During the second temperature ramp, the variation in the difference in power absorbed by the empty crucible and by the crucible containing the sample of wax is measured as a function of the temperature. The melting point of the compound is the temperature value corresponding to the top of the peak of the curve representing the variation in the difference in power absorbed as a function of the temperature.

The waxes can be hydrocarbon waxes, silicone waxes and/or fluoro waxes, and can be of plant, animal, mineral and/or synthetic origin.

Preferably, if present, the wax(es) are present in the composition in a content ranging from 0.01% to 5% by weight, preferably from 0.1% to 3% by weight, and preferably from 0.2% to 2% by weight, relative to the total weight of the composition, including all ranges and subranges therebetween.

According to preferred embodiments, the composition in accordance with the present disclosure comprises at least one wax of plant origin.

According to preferred embodiments, the composition in accordance with the present disclosure comprises at least one jojoba ester wax. The jojoba ester wax may be in an unsaturated form of jojoba esters, which may be the entirely hydrogenated jojoba ester having the formula below:

R¹—COO—CH₂—R¹ [chem 3]
in which:
R¹ is a CH₃—(CH₂)_{y} group, y is equal to 16, 18, 20 or 22.

The jojoba ester wax can be obtained by hydrogenation of a jojoba wax ester (jojoba oil) having the formula below:

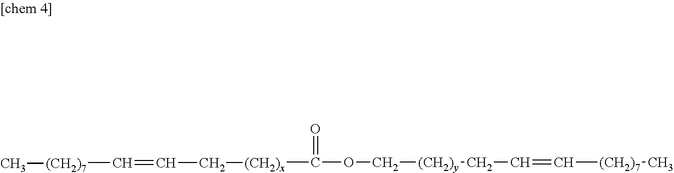

in which x and y are equal to 6, 8, 10 or 12.

The jojoba wax esters are composed of straight-chain monounsaturated fatty alcohols and of monounsaturated fatty acids. The only double bond is located in the middle (position n-9), starting from the methyl end group (—CH₃) of the chain of the respective fatty acid or alcohol.

Such wax esters are composed of fatty alcohols and of fatty esters with an even number of carbon atoms, mainly 20 and 22 carbon atoms. The resulting material comprises esters having chain lengths of 38, 40, 42 and 44 carbon atoms, with a small amount of esters of 36 and 46 carbon atoms being present. The typical composition of the wax esters is indicated below.

TABLE 1
Wax esters

| | | typical % (surface by |
|---|---|---|
| (X, Y) | Chain length | GPC) |
| (6, 6) | 36 | 1 |
| (6, 8) (8, 6) | 38 | 8 |
| (6, 10) (8, 8) (10, | 40 | 39 |
| 6) | | |
| (10, 8) (8, 10) | 42 | 38 |
| (10, 10) | 44 | 13 |
| (12, 10) (10, 12) | 46 | 1 |

The jojoba wax ester can be derived from the seed of the jojoba plant (Simmondsia chinensis).

By way of examples of a jojoba wax ester, mention may be made of the product sold under the name Floraesters® 70 by International Flora Technologies Ltd, and the product sold under the name Jojoba Esters-70 by Vantage.

Preferably, if present, the jojoba ester wax is present in the composition in an amount ranging from 0.1% to 2% by weight, and preferably from 0.2% to 0.8% by weight relative to the total weight of the composition, including all ranges and subranges therebetween.

According to preferred embodiments, the composition in accordance with the present disclosure comprises at least one additional hydrocarbon-based wax in addition to the jojoba ester mentioned above. Suitable examples include, but are not limited to, beeswax, lanolin wax or Chinese insect wax, rice wax, carnauba wax, candelilla wax, ouricury wax, esparto grass wax, cork fibre wax, sugar cane wax, Japan wax, Berry wax, shellac wax and sumac wax, Helianthus annuus (sunflower) seed wax, montan wax, microcrystalline waxes, paraffins and ozokerite; use may in particular be made of polyethylene waxes, polymethylene waxes, waxes obtained by Fischer-Tropsch synthesis and waxy copolymers, and also esters thereof. Mention may in particular be made of beeswax, for example the product sold under the name White beeswax BR G889 by Koster Keunen, carnauba wax, for example sold under the name Cerauba T1 Bio by Baerlocher, Helianthus annuus (sunflower) wax sold under the name Sunflower Wax by Koster Keunen, or a mixture thereof. Preferably, compositions of the present disclosure comprise Helianthus annuus (sunflower) seed wax.

If present, the additional hydrocarbon-based wax is preferably present in the composition in an amount ranging from 0.05% to 5% by weight, preferably from 0.1% to 1% by weight and preferably from 0.1% to 0.5% by weight, relative to the total weight of the composition, including all ranges and subranges therebetween.

According to preferred embodiments, the composition in accordance with the present disclosure comprises a mixture of glyceryl stearate, sodium steroyl glutamate, cetearyl alcohol, jojoba esters and sunflower seed wax, having the INCI name Cetearyl Alcohol (and) Jojoba Esters (and) Glyceryl Stearate (and) Sodium Stearoyl Glutamate (and) Helianthus Annuus Seed Cera (and) Polyglycerin-3, sold under the name Emulium® Dolcea MB by Gattefosse.

According to preferred embodiments, the composition in accordance with the present disclosure comprises:
bemotrizinol;
at least one semi-crystalline polymer as defined previously;
at least one salt chelating agent;
at least one C12-C24 saturated fatty alcohol;
at least one emulsifying silicone elastomer;
at least one carboxylic or carboxylate anionic surfactant chosen from amino acids modified with at least one C8-C24 hydrocarbon-based chain, and salts thereof; and
at least one wax.

According to preferred embodiments, the composition in accordance with the present disclosure comprises:
bemotrizinol;
at least one semi-crystalline polymer as defined previously;
at least one salt chelating agent;
at least one C12-C24 saturated fatty alcohol;
at least one emulsifying silicone elastomer;
at least one carboxylic or carboxylate anionic surfactant chosen from amino acids modified with at least one C8-C24 hydrocarbon-based chain, and salts thereof; and
at least one active agent.

According to preferred embodiments, the composition in accordance with the present disclosure comprises:
bemotrizinol;
at least one semi-crystalline polymer as defined previously;
at least one salt chelating agent;
at least one C12-C24 saturated fatty alcohol;
at least one emulsifying silicone elastomer;
at least one acyl glutamic acid (INCI name: acyl glutamic acid) or a salt thereof (acyl glutamates), the acyl group being a C10-C30, preferably C12-C22, acyl group; and
at least one coloring agent.

According to preferred embodiments, the composition in accordance with the present disclosure is a fluid composition for skin care, in the form of an oil-in-water emulsion, comprising, relative to the total weight of the composition:
(i) bemotrizinol;
(ii) from 0.05% to 10% by weight of a semi-crystalline polymer comprising monomer units of formulae (A) and (B):

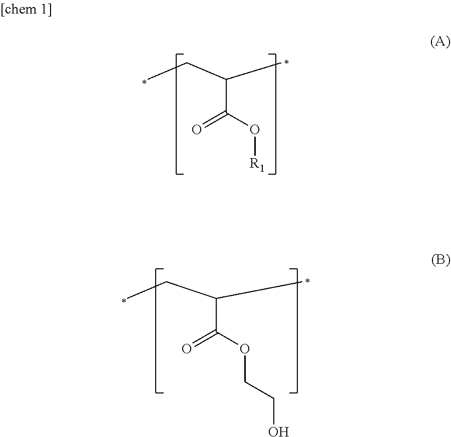

in which:
R1, independently at each instance, is chosen from alkyl or alkenyl radicals, and
at least 60% by weight of the R1 groups are radicals chosen from stearyl and behenyl radicals, the percentage by weight relating to the sum of all the R1 groups present in the polymer, and
the weight ratio of the sum of all the hydroxyethyl acrylate units to the sum of all the acrylate units bearing the R1 group ranges from 1:30 to 1:1,
and the sum of the total of units A and B is at least 95% by weight relative to the total weight of the polymer;
at least one salt chelating agent;
   at least one C12-C24 saturated fatty alcohol;
   at least one emulsifying silicone elastomer; and
   at least one acyl glutamic acid (INCI name: acyl glutamic acid) or a salt thereof (acyl glutamates), the acyl group being a C10-C30, preferably C12-C22, acyl group.

### Oil

According to preferred embodiments of the present disclosure, compositions further comprising at least one oil are provided. “Oil” means a substance which is hydrophobic and lipophilic, and is a liquid at about room temperature (20 to 25° C.) and about atmospheric pressure (760 mm Hg).

Suitable include volatile and/or non-volatile oils. Such oils can be any acceptable oil including but not limited to silicone oils and/or hydrocarbon oils.

According to certain embodiments, the compositions of the present disclosure preferably comprise one or more volatile silicone oils. Examples of such volatile silicone oils include linear or cyclic silicone oils having from 2 to 7 silicon atoms, these silicones being optionally substituted with alkyl or alkoxy groups of 1 to 10 carbon atoms. Specific oils that may be used in the disclosure include octamethyltetrasiloxane, decamethylcyclopentasiloxane, dodecamethylcyclohexasiloxane, heptamethyloctyltrisiloxane, hexamethyldisiloxane, decamethyltetrasiloxane, dodecamethylpentasiloxane and their mixtures. Other volatile oils which may be used include KF 96A of 6 cSt viscosity, a commercial product from Shin Etsu having a flash point of 94° C. Preferably, the volatile silicone oils have a flash point of at least 40° C.

Non-limiting examples of volatile silicone oils are listed in Table 1 below.

TABLE 1

| | Flash Point | Viscosity |
|---|---|---|
| Compound | (° C.) | (cSt) |
| Octyltrimethicone | 93 | 1.2 |
| Hexyltrimethicone | 79 | 1.2 |
| Decamethylcyclopentasiloxane | 72 | 4.2 |
| (cyclopentasiloxane or D5) | | |
| Octamethylcyclotetrasiloxane | 55 | 2.5 |
| (cyclotetradimethylsiloxane or D4) | | |
| Dodecamethylcyclohexasiloxane (D6) | 93 | 7 |
| Decamethyltetrasiloxane(L4) | 63 | 1.7 |
| KF-96 A from Shin Etsu | 94 | 6 |
| PDMS (polydimethylsiloxane) DC 200 | 56 | 1.5 |
| (1.5 cSt) from Dow Corning | | |
| PDMS DC 200 (2 cSt) from Dow Corning | 87 | 2 |

Further, a volatile linear silicone oil may be employed in the present disclosure. Suitable volatile linear silicone oils include those described in U.S. Pat. No. 6,338,839 and WO03/042221, the contents of which are incorporated herein by reference. In one embodiment the volatile linear silicone oil is decamethyltetrasiloxane. In another embodiment, the decamethyltetrasiloxane is further combined with another solvent that is more volatile than decamethyltetrasiloxane.

According to certain embodiments of the present disclosure, the composition of preferably comprises one or more non-silicone volatile oils and may be selected from volatile hydrocarbon oils, volatile esters and volatile ethers. Examples of such volatile non-silicone oils include, but are not limited to, volatile hydrocarbon oils having from 8 to 16 carbon atoms and their mixtures and in particular branched C₈ to C₁₆ alkanes such as C₈ to C₁₆ isoalkanes (also known as isoparaffins), isohexacecane, isododecane, isodecane, and for example, the oils sold under the trade names of Isopar or Permethyl. Preferably, the volatile non-silicone oils have a flash point of at least 40° C.

Non-limiting examples of volatile non-silicone volatile oils are given in Table 2 below.

TABLE 2

| Compound | Flash Point (° C.) |
|---|---|
| Isododecane | 43 |
| Propylene glycol n-butyl ether | 60 |
| Ethyl 3-ethoxypropionate | 58 |
| Propylene glycol methylether acetate | 46 |
| Isopar L (isoparaffin C₁₁-C₁₃) | 62 |
| Isopar H (isoparaffin C₁₁-C₁₂) | 56 |

According to certain embodiments of the present disclosure, the composition comprises at least one non-volatile oil. Examples of non-volatile oils that may be used in the present disclosure include, but are not limited to, polar oils such as, for example:
esters and ethers, in particular fatty acids, such as oils of formulas R1 COOR2, wherein R 1 represents the remainder of a fatty acid having from 8 to 29 carbon atoms, and R 2 represents a hydrocarbon chain, branched or not, containing from 3 to 30 carbon atoms, such as for example Purcellin oil, isononyl isononanoate, isopropyl myristate, ethyl-2-hexyl palmitate, octyl-2-dodecyl stearate, octyl-2-dodecyl erucate, isostearyl isostearate; hydroxylated esters such as isostearyl lactate, octylhydroxystearate, octyldodecyl hydroxystearate, diisostearylmalate, triisocetyl citrate, heptanoates, octanoates, fatty alcohol decanoates; polyol esters, such as propylene glycol dioctanoate, neopentylglycol diheptanoate and diethylene glycol diisononanoate; tryiglycerides such as caprylic/capric acid triglycerides, for instance those sold by the company Stearineries Dubois or those sold under the names Miglyol 810, 812 and 818 by the company Dynamit Nobel; and esters of pentaerythritol such as pentaerythrityl tetraisostearate or dipentaerythrityl pentaisononanoate;
ethers containing from 10 to 40 carbon atoms;
liquid C₈ to C₂₆ fatty alcohols, for instance oleyl alcohol, cetyl alcohol, stearyl alcohol, octyldodecanol, and cetearly alcohol;
hydrocarbon oils of animal origin, such as perhydrosqualene;
hydrocarbon oils of vegetable origin, such as liquid triglycerides of fatty acids having from 4 to 10 carbon atoms such as triglycerides of heptanoic or octanoic acids or, for example, sunflower, corn, soybean, squash, grape seed, sesame, hazelnut, apricot, macadamia, ara, sunflower oil, castor oil, avocado, triglycerides of caprylic/capric acids such as those sold by the company Stearineries Dubois or those sold under the names Miglyol 810, 812 and 818 by the company Dynamit Nobel, coco-caprylate/caprate (esterified oil from coconut oil), jojoba oil, shea butter oil; and
mixtures thereof.

Further, examples of non-volatile oils that may be used in the present disclosure include, but are not limited to, non-polar oils such as branched and unbranched hydrocarbons, in particular Vaseline (petrolatum), paraffin oil, squalane, squalene, hydrogenated polyisobutene, hydrogenated polydecene, polybutene, mineral oil, pentahydrosqualene, and mixtures thereof.

According to certain embodiments of the present disclosure, the compositions of the present disclosure comprise at least one non-volatile silicone oil. Suitable examples of such silicone oils include, but are not limited to, non-volatile silicone fluids such as, for example, polyalkyl (aryl) siloxanes. Suitable polyalkyl siloxanes include, but are not limited to, polydimethyl siloxanes, which have the CTFA designation dimethicone, polydiethyl siloxane, phenyl trimethicone, trimethyl pentaphenyl trisiloxane, phenyldimethicone, phenyltrimethylsiloxydiphenylsiloxane, diphenyldimethicone, and diphenylmethyldiphenyltrisiloxane and those siloxanes disclosed in U.S. patent application publication no. 2004/0126350, the entire disclosure of which is hereby incorporated by reference. Specific examples of suitable high viscosity silicone oils include, but are not limited to, 15 M 30 from PCR (500 cSt) or Belsil PDM 1000 (1 000 cSt) from Wacker and Dow Corning 200 (350 cSt) (the values in parenthesis represent viscosities at 25° C.).

Particularly preferred oils include, but are not limited to, one or more of the following: Diisopropyl Sebacate, C12-15 Alkyl Benzoate, Phenethyl Benzoate, Isopropyl Lauroyl Sarcosinate, Diisopropyl Adipate, Dibutyl Adipate, Dicaprylyl Carbonate, Dicaprylate/Dicaprate, Coco Glycerides, Caprylic/capric triglyceride, Isopropyl Myristat, Isopropyl Palmitate, Coco Caprylate/Caprate, Ethylhexyl Palmitate, Isononyl isononanoate, Octyl dodecanol, Isohexadecane, isododecane, Dicaprylyl Ether, C15-19 Alkane, and mxtures thereof.

According to preferred embodiments, the at least one oil is/are present in the compositions of the present disclosure in an amount ranging from about 1% to about 50% by weight, more preferably from about 5 to about 40% by weight, and preferably from about 10% to about 35% by weight, based on the total weight of the composition, including all ranges and subranges within these ranges such as, for example, 15% to 40%, 20% to 45%, etc.

According to preferred embodiments, the oil phase comprises of the emulsion compositions of the present disclosure is present in an amount ranging from about 20% to about 50% by weight, more preferably from about 25 to about 45% by weight, and preferably from about 30% to about 40% by weight, based on the total weight of the composition, including all ranges and subranges within these ranges such as, for example, 25% to 40%, 20% to 45%, etc.

### Aqueous Phase

According to the present disclosure, compositions comprising an aquouse phase comprising water are provided. Preferably, when the compositions of the present disclosure contain water, they are in the form of an emulsion containing an external aqueous phase such as an oil-in-water emulsion (O/W) or a water-in-oil-in-water emulsion (O/W/O). Preferably, when in the form of an emulsion, the oil phase can contain silicone oils (e.g., Si/W or W/Si emulsion) or hydrocarbon oils. When present, water is preferably present in an amount of from about 10% to about 80% by weight, preferably from about 20% to about 70% by weight, preferably from about 35% to about 65% by weight, including all ranges and subranges therebetween, all weights being based on the total weight of the composition.

The aqueous phase may optionally further comprise at least one water-soluble organic solvent which is liquid at room temperature and atmospheric pressure. For example, such at least one water-soluble organic solvent may include:
Triethyl citrate;
C1-C5 monoalcohols having a C1-C5 alkane chain and a single hydroxyl function (OH). Suitable C1-C5 monoalcohols include methanol, ethanol, propanol, isopropanol, butanol and mixtures thereof;
polyols (compounds having 2 or more hydroxyl groups) having, for example from 2 to 20 carbon atoms, preferably from 2 to 6 carbon atoms, such as for example glycerol, diglycerol, propylene glycol, isoprene glycol, dipropylene glycol, butylene glycol, hexylene glycol, 1,3-propanediol, pentylene glycol, simple sugars, and water-soluble polyalkylene glycols;
and mixtures thereof.

According to preferred embodiments, the at least one water-soluble organic solvent is selected from the group consisting of ethanol, dipropylene glycol, butylene glycol, propanediol and propylene glycol, and mixtures thereof.

If present, the water-soluble organic solvent(s) is/are preferably present in compositions of the present disclosure in an amount ranging from about 0.5 to about 40% by weight, preferably from about 3 to about 30% by weight, and preferably from about 5% to about 20% by weight relative to the total weight of the composition, including all ranges and subranges therebetween such as, for example, 2% to 15%, 2% to 25%, 7.5% to 30%, etc.

According to preferred embodiments, compositions of the present disclosure comprise at least polyol, preferably at least one polyol selected from the group consisting of glycerol, diglycerol, propylene glycol, isoprene glycol, dipropylene glycol, butylene glycol, hexylene glycol, 1,3-propanediol, pentylene glycol, and mixtures thereof. If present, the at least one polyol is preferably present in compositions of the present disclosure in an amount ranging from about 0.1 to about 10% by weight, preferably from about 0.5 to about 5% by weight, and preferably from about 1% to about 3% by weight relative to the total weight of the composition, including all ranges and subranges therebetween such as, for example, 2% to 3%, 1.2% to 2,5%, 0.75% to 3%, etc.

### Additional Optional Ingredients

Compositions of the present disclosure may also optionally further include at least one additive or auxiliary commonly used in cosmetic compositions and known to a person skilled in the art as being capable of being incorporated into such compositions. Such additives or auxiliaries may be chosen from additional film formers, additional surfactants, salt chelating agent, natural gums, elastomers, coloring agents (e.g., dyes and pigments), waxes, thixotropic agents (e.g., clays), fillers, preservatives, fragrances, antioxidants, agents for combating free radicals, spreading agents, dispersing agents, antifoaming agents, neutralizing agents, stabilizing agents, active principles chosen from essential oils, moisturizing agents, vitamins, actives, proteins, ceramides, plant extracts, fibers, and the like, wetting agents and their mixtures. Although, preferably, compositions of the present disclosure are “free of,” “substantially free of,” or “devoid of” such additives.

A person skilled in the art will take care to select the optional additional additives and/or the amount thereof such that the advantageous properties of the composition according to the disclosure are not, or are not substantially, adversely affected by the envisaged addition.

Needless to say, the composition of the disclosure should be cosmetically or dermatologically acceptable, i.e., it should contain a non-toxic physiologically acceptable. The composition may be in any galenic form normally employed in the cosmetic and dermatological fields which is suitable for topical administration as discussed above.

These auxiliary additives may be present in the composition in a proportion from 0% to 99% (such as from 0.01% to 90%) relative to the total weight of the composition and further such as from 0.1% to 50% (if present), including all ranges and subranges therebetween.

### Natural Gum

According to preferred embodiments, compositions of the present disclosure may optionally further comprise at least one natural gum. Preferred water-soluble natural gums include, but are not limited to, guar, acacia (Senegal) gum, xanthan, gum arabic, gum tragacanth, locust bean bum, agar, sclerotium gum, and mixtures thereof. Preferably, if present, the at least one natural gum is present in an amount of from about 0.05% to 10% by weight, preferably from about 0.075% to about 5% by weight, preferably from about 0.1% to about 1% by weight, and preferably from about 0.1% to about 0.5% by weight relative to the total weight of the composition, including all ranges and subranges therebetween.

### Salt Chelating Agent

According to preferred embodiments, compositions of the present disclosure may optionally further comprise at least one salt chelating agent. “Salt” chelating agents are preferably chosen from alkali metal, alkaline-earth metal, and/or ammonium salts of chelating agents.

Suitable chelating agents include, but are not limited to, aminocarboxylic acids (i.e. acids comprising at least one carboxylic acid group) such as, for example, phytic acid, diethylenetriaminepentaacetic acid (DTPA), ethylenediaminedisuccinic acid (EDDS) and trisodium ethylenediamine disuccinate such as Octaquest E30 from Octel, ethylenediaminetetraacetic acid (EDTA), ethylenediamine-N,N′-diglutaric acid (EDDG), glycinamide-N,N′-disuccinic acid (GADS), 2-hydroxypropylenediamine-N,N′-disuccinic acid (HPDDS), ethylenediamine-N,N′-bis(ortho-hydroxyphenylacetic acid) (EDDHA), N,N′-bis(2-hydroxybenzyl)ethylenediamine-N,N′-diacetic acid (HBED), nitrilotriacetic acid (NTA), methylglycine diacetic acid (MGDA), N-2-hydroxyethyl-N,N-diacetic acid and glyceryl imino diacetic acid (as described in documents EP-A-317 542 and EP-A-399 133), iminodiacetic acid-N-2-hydroxypropyl sulfonic acid and aspartic acid N-carboxymethyl N-2-hydroxypropyl-3-sulfonic acid (as described in EP-A-516 102), beta-alanine-N,N′-diacetic acid, aspartic acid-N,N′-diacetic acid and aspartic acid-N-monoacetic acid (described in EP-A-509 382), chelating agents based on iminodisuccinic acid (IDSA) (as described in EP-A-509 382), ethanoldiglycine acid, phosphonobutane tricarboxylic acid, such as the compound sold by Bayer under the reference Bayhibit AM, tetrasodium glutamate diacetate (GLDA) such as Dissolvine GL38 or 45S from Akzo Nobel, chelating agents based on mono- or polyphosphonic acid, such as diethylenetriaminepenta (methylenephosphonic acid) (DTPMP), ethane-1-hydroxy-1,1,2-triphosphonic acid (E1 HTP), ethane-2-hydroxy-1,1,2-triphosphonic acid (E2HTP), ethane-1-hydroxy-1,1-diphosphonic acid (EHDP), ethane-1,1,2-triphosphonic acid (ETP), ethylenediaminetetramethylenephosphonic acid (EDTMP), hydroxyethane-1,1-diphosphonic acid (HEDP), chelating agents based on polyphosphoric acid, such sodium tripolyphosphate (STP), tetrasodium diphosphate, hexametaphosphoric acid, sodium metaphosphate, phytic acid, Among the chelating agents mentioned, ethylenediamine-tetraacetic acid (EDTA), diethylenetriaminepentaacetic acid (DTPA), S,S′-ethylenediaminedisuccinic acid (EDDS), ethylenediaminetetramethylenephosphonic acid (EDTMP), phosphonobutanetricarboxylic acid and tetrasodium glutamate diacetate (GLDA), and mixtures thereof, are preferably used. The chelating agent may be selected from the group consisting of dipotassium ethylenediamine tetraacetic acid (EDTA), disodium EDTA, tetrasodium EDTA, diammonium EDTA, calcium disodium EDTA, triethanolamine EDTA, tripotassium EDTA, trisodium EDTA, hydroxyethyl ethylenediamine triacetic acid (HEDTA), trisodium HEDTA, trisodium ethylenediamine disuccinate, and combinations thereof. In one embodiment, the chelating agent comprises tetrasodium EDTA. Tetrasodium Glutamate Diacetate (e.g., Dissolvine GL-47-S), which is a high purity, versatile and readily biodegradable chelate based upon L-glutamic acid, a natural and renewable raw material, can be used in the disclosed formulation. Ideally, the percentage range for the chelating agent in the disclosed formulation is between 0.01-1.00% for optimal result. The chelating agent may be present in an amount from about 0.001% to about 2% by weight relative to the total weight of the composition, for example from about 0.001% to about 1.5%, or from about 0.005% to about 1%, or from about 0.008% to about 0.8%, or from about 0.01% to about 0.5%, or from about 0.01% to about 0.25%. In one embodiment, the chelating agent is present in amount from about 0.02% to about 0.2%.

In accordance with the present disclosure, compositions of the present disclosure can be a stand-alone product (for use by itself), or they can be a product for use in conjunction with another composition, for example it can be a basecoat (primer) composition, a color coat composition, or a topcoat (over coat) composition. It should be understood that when compositions of the present disclosure are applied to keratinous materials in the form of any of such compositions, such application can comprise one or more layers of the product. Thus, for example, application of at least one color coat composition can comprise one or more color coat layers; application of the at least topcoat composition can comprise one or more topcoat layers; and application of the at least one basecoat composition can comprise one or more basecoat layers. Preferably, such basecoat, color coat and topcoat compositions contain three or fewer layers of compositions, preferably two or fewer layers of compositions, and preferably a single layer of compositions.

During application of compositions of the present disclosure, basecoat (if present) is typically applied directly to keratinous material, color coat is typically applied either directly to the keratinous material (if no basecoat is present) or to a previously-applied basecoat, and topcoat (if present) is typically applied to a color coat.

According to preferred embodiments of the present disclosure, methods of treating, protecting, enhancing the appearance of, caring for and/or making up keratinous material by applying compositions of the present disclosure to the keratinous material in an amount sufficient to treat, enhance the appearance of, care for and/or make up the keratinous material are provided.

Preferably, “making up” keratinous material includes applying a composition comprising at least one coloring agent to the keratinous material in an amount sufficient to provide color and/or optical effect to the keratinous material.

Preferably, “protecting” keratinous material includes applying a composition of the present disclosure to protect keratinous material from damage resulting from exposure to UV rays.

In accordance with the preceding embodiments, compositions of the present disclosure are applied topically to the keratinous material in an amount sufficient to treat, enhance the appearance of, care for and/or make up the keratinous material. The compositions may be applied to the desired area as needed, preferably once or twice daily, more preferably once daily and then preferably allowed to dry before subjecting to contact such as with clothing or other objects (for example, clothes or a topcoat). Preferably, the composition is allowed to dry for about 1 minute or less, more preferably for about 45 seconds or less.

The present disclosure also relates to methods of making compositions comprising combining bis-ethylhexyloxyphenol methoxyphenyl triazine and at least one semi-crystalline polymer during formation of the compositions. Preferably, the compositions are produced in the form of an oil-in-water emulsion. Preferably, the methods further comprise adding at least one surfactant selected from the group consisting of alkylpolyglucosides, polyol fatty acid esters, anionic carboxylic surfactants or carboxylates, and mixtures thereof, during formation of the compositions, preferably emulsion compositions. Preferably, the compositions further comprise at least one salt chelating agent.

The present disclosure also envisages kits and/or prepackaged materials suitable for consumer use containing one or more compositions according to the description herein, alone or in combination with other consumer care products such as makeup products such as basecoats, topcoats, removal compositions, etc. The packaging and application device for any subject of the disclosure may be chosen and manufactured by persons skilled in the art on the basis of their general knowledge, and adapted according to the nature of the composition to be packaged. Indeed, the type of device to be used can be in particular linked to the consistency of the composition, in particular to its viscosity; it can also depend on the nature of the constituents present in the composition, such as the presence of volatile compounds.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present disclosure.

Notwithstanding that the numerical ranges and parameters setting forth the broad scope of the disclosure are approximations, the numerical values set forth in the specific examples are reported as precisely as possible. Any numerical value, however, inherently contain certain errors necessarily resulting from the standard deviation found in their respective measurements. The following examples are intended to illustrate the disclosure without limiting the scope as a result. The percentages are given on a weight basis.

### Example 1—Liquid Serum

| | % |
|---|---|
| Ingredient | Concentration |
| WATER/AQUA | QS |
| CHELATING AGENT | 0.3 |
| PRESERVATIVE | 1 |
| POLYOL | 3 |
| FATTY ALCOHOL/SURFACTANT | 2 |
| NATURAL GUM | 0.1 |
| BUTYL METHOXYDIBENZOYLMETHANE | 3 |
| HOMOSALATE/OCTISALATE/OCTOCRYLENE | 22 |
| BIS-ETHYLHEXYLOXYPHENOL | 2 |
| METHOXYPHENYL TRIAZINE | |
| C12-22 ALKYL ACRYLATE/ | 1 |
| HYDROXYETHYLACRYLATE COPOLYMER | |
| NONVOLATILE SOLVENT | 6 |
| ACTIVE AGENT | 1 |

What is claimed is:

1. A composition comprising bemotrizinol, at least one salt chelating agent, and at least one semi-crystalline polymer comprising at least one alkyl acrylate chain.

2. The composition of claim 1, wherein the at least one semi-crystalline polymer comprising at least one alkyl acrylate chain is/are selected from the group consisting of homopolymers obtained by polymerization of a monomer chosen from alkyl acrylates and alkyl methacrylates, copolymers obtained by copolymerization of a monomer chosen from alkyl acrylates and alkyl methacrylates, with a hydrophilic monomer, and mixtures thereof.

3. The composition of claim 1, wherein the at least one semi semi-crystalline polymer comprising at least one alkyl acrylate chain is/are selected from the group consisting of a stearyl polyacrylate, a polyacrylate of behenyl, a behenyl acrylate/2-hydroxyethyl acrylate copolymer, a stearyl acrylate/2-hydroxyethyl acrylate copolymer, and mixtures thereof.

4. The composition of claim 1, wherein the composition has one or more of the following properties:
a critical wavelength of at least 370 nm;
a UVAPF/SPF ratio of at least 1/3; and
a UVA1/UV ratio of 0.7 or higher.

5. The composition of claim 4, wherein the composition has two or more of the properties, preferably all three of the properties.

6. The composition of claim 4, wherein the composition also has an SPF value of at least 30.

7. The composition of claim 1, wherein the composition is in the form of an oil-in-water emulsion.

8. The composition of claim 1, further comprising at least one surfactant selected from the group consisting of at least one emulsifying silicone elastomerS.

9. The composition of claim 1, wherein the composition comprises 10% or less by weight relative to the total weight of composition of additional organic UV filters.

10. The composition of claim 1, wherein the composition is free of oxybenzone and/or octinoxate.

11. The composition of claim 1, further comprising at least one active agent.

12. The composition of claim 1, wherein the pH is 5 or greater.
